(19) United States
(12) Patent Application Publication
(10) Pub. No.: US 2010/0062421 A1
(43) Pub. Date: Mar. 11, 2010

(54) COMPOSITIONS, METHODS, AND DEVICES FOR ISOLATING BIOLOGICAL MATERIALS

(76) Inventors: Wensheng Xia, Woodbury, MN (US); Paul N. Holt, Hudson, WI (US); Ranjani V. Parthasarathy, Woodbury, MN (US); Manjiri T. Kshirsagar, Woodbury, MN (US)

Correspondence Address: 3M INNOVATIVE PROPERTIES COMPANY, PO BOX 33427, ST. PAUL, MN, 55133-3427, US

(21) Appl. No.: 12/597,326

(22) PCT Filed: Apr. 25, 2008
(86) PCT No.: PCT/US08/61508

§ 371 Date: Oct. 23, 2009

Related U.S. Application Data
(60) Provisional application No. 60/913,812, filed on Apr. 25, 2007.

(51) Int. Cl. C12Q 1/70; C07H 21/02; C12Q 1/68; C12M 1/33; C12N 7/02; C12N 1/06; C12N 1/20

(52) U.S. Cl. 435/5; 536/23.1; 536/25.4; 435/6; 435/306.1; 435/239; 435/255.1; 435/254.1; 435/252.1

(57) ABSTRACT

Compositions, methods, devices, and kits, which include an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and a lanthanide; y is an integer from 3 to 6; and x is 1 or 2, and to which microorganisms and polynucleotides bind, and which can be used for separating and optionally assaying microorganisms and/or a polynucleotide from a sample material are disclosed.

This application claims the benefit of U.S. Provisional Application No. 60/913,812, filed Apr. 25, 2007, which is incorporated herein by reference in its entirety.

### BACKGROUND

Isolating a biological material, for example, cells, viruses, and polynucleotides, from a sample can be helpful or even necessary when applying a method for detecting or assaying the biological material. In some methods, microorganisms are isolated from a sample, and enumerative or non-enumerative methods are used to determine total numbers of microorganisms or to identify at least some of the microorganisms. Standard Plate Count, coliform, yeast and mold counts, bioluminescence assays and impedance or conductance measurements for enumeration and selective and differential plating, DNA hybridization, agglutination, and enzyme immunoassay for non-enumeration, for example, have been used. Identification of a polynucleotide or a portion of a polynucleotide has been used for diagnosing a microbial infection, detecting genetic variations, typing tissue, and so on. Methods for identifying polynucleotides, including DNA and RNA, often include amplifying or hybridizing the polynucleotide. Examples of amplification methods include polymerase chain reaction (PCR); target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT), nucleic acid sequence-based amplification (NASBA), amplification under the trade name INVADER, and transcription-mediated amplification (TMA); and various other amplification methods, such as repair chain reaction (RCR) and cycling probe reaction (CPR). Separating polynucleotides from a sample, which is often a complex mixture, can be necessary because large amounts of cellular or other contaminating material such as carbohydrates and proteins can interfere with these methods.

Methods are known for isolating polynucleotides from a sample. Some of these involve a time consuming series of extraction and washing steps. For example, nucleic acids have been isolated from a sample, such as a blood sample or a tissue sample, by lysis of the biological material using a detergent or chaotrope, extractions with organic solvents, precipitation with ethanol, centrifugations, and dialysis of the nucleic acid.

Solid extraction has also been employed in certain methods of isolating nucleic acids. Here the uses of particles, including microbeads, and membrane filters have been practiced. For example, DNA extraction has been carried out by absorption of DNA onto silica particles under chaotropic conditions. However, a subsequent washing step typically requires an organic solvent such as ethanol or isopropanol. Other examples of such methods have been reported, which include utilizing a hydrophobic surface in the presence of certain surfactants or polyethylene glycol, together with a high concentration of a salt. The use of organic solvents or high concentrations of salt limits the versatility of the extraction method for combining with subsequent methods such as nucleic acid amplification in microfluidic systems. Moreover, the use of multiple reagents during the extraction process is costly and time consuming. In another example, ammonium groups bound to a surface are used to attract and bind DNA molecules. DNA extraction kits having this capability are available, for example, from Qiagen (Valencia, Calif.). Eluting the adsorbed DNA is normally done at high pH or high concentration of salt, which can interfere with subsequent methods such as DNA amplification. Significant dilutions of the acquired material which can result in reduced sensitivity, or de-salting, or neutralization may be required.

An immobilized metal affinity chromatography (IMAC) method for separating and/or purifying compounds containing a non-shielded purine or pyrimidine moiety or group, such as nucleic acid, has been reported (U.S. Publication No. 2004/0152076A1). However, double stranded DNA was found not bind to the column matrix.

With the growing importance of improved sample preparation methods and detecting microorganisms, there is a continuing need for materials and methods for isolating microorganisms and/or which are simple enough to extract polynucleotides under mild conditions and sufficiently versatile to be used with subsequent methods without interfering with such methods, or which may provide value by reducing labor.

### SUMMARY OF THE INVENTION

It has now been found that polynucleotides, including double stranded DNA, can be isolated from complex sample material using certain immobilized-metal support materials. Although not wishing to be bound by theory, Applicants believe that certain metal ions bound to the support material interact with phosphate groups on the polynucleotides, causing the polynucleotides to bind to the immobilized-metal support material. Moreover, the captured polynucleotides can be released with a short period of moderate heating and with a low concentration of a buffer which competes with or displaces the polynucleotide phosphate groups. The released polynucleotide in combination with the buffer can be used directly for downstream processes such as polynucleotide amplification.

It has also been found that the immobilized-metal support materials non-specifically bind microorganisms, which can then be isolated from sample materials, including complex samples such food and clinical samples. “Non-specifically binding” means that the binding is not specific to any type of microorganism or bacterial cell or the like. Thus, for example, all bacteria in a sample can be isolated from other components in the sample rather than targeting, for example, one strain of bacteria. Both gram positive and gram negative bacteria, yeast cells, mold spores, and the like can be bound. The resulting isolated microorganisms can then be subjected to known detection methods, such as microorganism load detection.

Accordingly, in one embodiment, the present invention provides a composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one double stranded polynucleotide bound to at least one of the metal ions, M^{y+};

wherein M is selected from the group consisting of zirconium, gallium, iron, aluminum, scandium, titanium, vanadium, yttrium, and a lanthanide; y is an integer from 3 to 6; and x is 1 or 2.

In another embodiment, the present invention provides a composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one polynucleotide bound to at least one of the metal ions, M^{y+};

wherein M is selected from the group consisting of zirconium, gallium, iron, aluminum, scandium, titanium, vanadium, yttrium, and a lanthanide; y is an integer from 3 to 6; and x is 1 or 2; and

wherein the composition has a pH of 4.5 to 6.5.

In another embodiment, the present invention provides a method of separating and optionally assaying at least one double stranded polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, M^{y+}, bound to the —C(O)O⁻ or —P(O)(—OH)₂₋ₓ(—O⁻)ₓ groups to provide a composition comprising a) the at least one double stranded polynucleotide bound to the immobilized-metal support material and b) a supernate comprising the sample material having a reduced amount of the at least one double stranded polynucleotide;

wherein M is selected from the group consisting of zirconium, gallium, iron, aluminum, scandium, titanium, vanadium, yttrium, and a lanthanide; y is an integer from 3 to 6; and x is 1 or 2.

In another embodiment, the present invention provides a method of separating and optionally assaying at least one polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, M^{y+}, bound to the —C(O)O⁻ or —P(O)(—OH)₂₋ₓ(—O⁻)ₓ groups, at a pH of 4.5 to 6.5, to provide a composition comprising a) the at least one polynucleotide bound to the immobilized-metal support material and b) a supernate comprising the sample material having a reduced amount of the at least one polynucleotide;

wherein M is selected from the group consisting of zirconium, gallium, iron, aluminum, scandium, titanium, vanadium, yttrium, and a lanthanide; y is an integer from 3 to 6; and x is 1 or 2; and

wherein the composition has a pH of 4.5 to 6.5.

In another embodiment, the present invention provides a device for processing sample material, the device having:

at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and at least one second chamber separate from the first chamber and capable of receiving and containing the fluid, the immobilized-metal support material, or both from the at least one first chamber;

wherein M is selected from the group consisting of zirconium, gallium, iron, aluminum, scandium, titanium, vanadium, yttrium, and the lanthanides; y is an integer from 3 to 6; and x is 1 or 2.

In another embodiment, the present invention provides a kit for separating at least one polynucleotide from a sample material, the kit comprising:

a device having at least one chamber capable of containing or channeling a fluid;

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and the lanthanides; y is an integer from 3 to 6; and x is 1 or 2; and

at least one reagent selected from the group consisting of a lysis reagent, a lysis buffer, a binding buffer, a wash buffer, and an elution buffer.

In another embodiment, the present invention provides a kit for separating and optionally assaying at least one polynucleotide from a sample material, the kit comprising a device for processing sample material, the device having:

at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one second chamber separate from the first chamber and capable of receiving and containing the fluid, the immobilized-metal support material, or both from the at least one first chamber;

wherein M is selected from the group consisting of zirconium, gallium, iron, aluminum, scandium, titanium, vanadium, yttrium, and the lanthanides; y is an integer from 3 to 6; and x is 1 or 2.

In another embodiment, there is provided a composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

a plurality of microorganisms, selected from the group consisting of bacterial cells, yeast cells, mold cells, viruses, and a combination thereof, non-specifically bound to the immobilized-metal support material;

wherein M is selected from the group consisting of zirconium, gallium, iron, aluminum, scandium, titanium, vanadium, yttrium, and a lanthanide; y is an integer from 3 to 6; and x is 1 or 2.

In another embodiment, there is provided method of isolating bacterial cells comprising:

providing a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups;

providing a sample suspected of having a plurality of microorganisms selected from the group consisting of bacterial cells, yeast cells, mold cells, viruses, and a combination thereof;

contacting the composition with the sample; wherein at least a portion of the plurality of microorganisms from the sample become non-specifically bound to the immobilized-metal support material;

wherein M is selected from the group consisting of zirconium, gallium, iron, aluminum, scandium, titanium, vanadium, yttrium, and a lanthanide; y is an integer from 3 to 6; and x is 1 or 2.

The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., a pH of 7 to 10 includes a pH of 7, 7.5, 8.0, 8.7, 9.3, 10, etc.).

The above summary of the present invention is not intended to describe each disclosed embodiment or every implementation of the present invention. The description that follows more particularly exemplifies illustrative embodiments.

### BRIEF DESCRIPTIONS OF THE FIGURE

FIG. 1 is a top view of a device according to the present invention with two separate chambers and with the immobilized-metal support material in one of the chambers.

### DETAILED DESCRIPTION OF ILLUSTRATIVE EMBODIMENTS OF THE INVENTION

The present invention provides compositions, methods, devices, and kits that can be used for isolating microorganisms and/or a polynucleotide from a sample material. Optionally, the isolated polynucleotide or microorganisms can be assayed. Assaying includes detecting the presence of the polynucleotide and/or determining the quantity of the polynucleotide that is present. In the case of microorganisms, assaying includes detecting the presence of microorganisms (identifying) and/or enumerating the quantity of microorganisms that are present. As used herein the term “polynucleotide” refers to single and double stranded nucleic acids, oligonucleotides, compounds wherein a portion of the compound comprises an oligonucleotide or polynucleotide, and peptide nucleic acids (PNA), and includes linear and circular forms. For certain embodiments, the polynucleotide is preferably a single or double stranded nucleic acid.

In one embodiment, there is provided a composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one double stranded polynucleotide bound to at least one of the metal ions, M^{y+};

wherein M is selected from the group consisting of zirconium, gallium, iron, aluminum, scandium, titanium, vanadium, yttrium, and a lanthanide; y is an integer from 3 to 6; and x is 1 or 2.

As used herein, the term “substrate” refers to a material with a solid surface, which can be, for example, a plurality of particles, the interior walls of a column, a filter, a microtiter plate, a frit, a pipette tip, a film, a plurality of fibers, or a glass slide. For certain embodiments, the substrate is selected from the group consisting of interior walls of a column, a filter, a microplate, a microfilter plate, a microtiter plate, a frit, a pipette tip, a film, a plurality of microspheres, a plurality of fibers, and a glass slide. For certain embodiments, the substrate is selected from the group consisting of beads, a gel, a film, a sheet, a membrane, particles, fibers, a filter, a plate, a strip, a tube, a column, a well, a wall of a container, a capillary, a pipette tip, and a combination thereof. The plurality of particles or particles can be a plurality of microparticles, which include microspheres, microbeads, and the like. Such particles can be resin particles, for example, agarose, latex, polystyrene, nylon, polyacylamide, cellulose, polysaccharide, or a combination thereof, or inorganic particles, for example, silica, aluminum oxide, or a combination thereof. Such particles can be magnetic or non-magnetic. Such particles can be colloidal in size, for example about 100 nm to about 10 microns (μ).

The plurality of —C(O)O⁻ or —P(O)(—OH)₂₋ₓ(—O⁻)ₓ groups can be bound to the substrate in a number of ways. For example, the groups can be bound by covalent bonding, ionic bonding, hydrogen bonding, and/or van der Waals forces. The groups can be bound directly to the substrate, such as a substrate having a polymeric surface wherein a polymer has —C(O)O⁻ or —P(O)(—OH)₂₋ₓ(—O⁻)ₓ groups covalently bonded to the polymer chain. Polymers of this nature can include —C(O)OH or —P(O)(—OH)₂ substituted vinyl units, for example, acrylic acid, methacrylic acid, vinylphosphonic acid, and like units. The —C(O)O⁻ or —P(O)(—OH)₂₋ₓ(—O⁻)ₓ groups can be bound indirectly to the substrate through a connecting group. For example, amino groups on a substrate can be contacted with a compound having multiple carboxy groups, such as nitrilotriacetic acid, to form an amide-containing connecting group which attaches one or more carboxy groups (two carboxy groups in the case of nitrilotriacetic acid) to the substrate. Substrates having available amino groups or which can be modified to have available amino groups are known to those skilled in the art and include, for example, agarose-based, latex-based, polystyrene-based, and silica-based substrates. Silica-based substrates such as glass or silica particles having —Si—OH groups can be treated with known aminosilane coupling agents, such as 3-aminopropyltrimethoxysilane, to provide available amino groups. Functional groups such as —C(O)OH or —P(O)(—OH)₂ can be attached to a substrate, for example, a substrate having a silica surface, using other known silane compounds.

The —C(O)O⁻ or —P(O)(—OH)₂₋ₓ(—O⁻)ₓ groups can also be bound indirectly to the substrate under conditions where these groups are attached to a molecule which binds to the substrate by electrostatic, hydrogen bonding, coordination bonding, van der Waals forces (hydrophobic interaction) or specific chemistry such as biotin-avidine interaction. For example, polymers bearing C(O)O⁻ or —P(O)(—OH)₂₋ₓ(—O⁻)ₓ groups can be coated on a surface with opposite charge using a Layer-by-Layer technique to build up a high density of polymer having C(O)O⁻ or —P(O)(—OH)₂₋ₓ(—O⁻)ₓ groups.

For a further example, monomers bearing C(O)O⁻ or —P(O)(—OH)₂₋ₓ(—O⁻)ₓ groups can be grafted to a polymer surface through plasma treatment.

Substrates having a plurality of carboxyl groups, e.g., —C(O)OH or —C(O)O⁻, are known and commercially available. For example, carboxylated microparticles are available under trade names such as DYNABEADS MYONE (Invitrogen, Carlsbad, Calif.) and SERA-MAG (Thermo Scientific, known as Seradyn, Indianapolis, Ind.).

The metal ions, M^{y+}, can be bound to acid groups by contacting the acid groups with an excess of metal ions, for example, as a solution of the metal salt, such as a nitrate salt. Other salts may be used as well, for example, chloride, perchlorate, sulfate, phosphate, acetate, acetylacetonate, bromide, fluoride, or iodide, salts.

In another embodiment, there is provided a composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one polynucleotide bound to at least one of the metal ions, M^{y+};

wherein M is selected from the group consisting of zirconium, gallium, iron, aluminum, scandium, titanium, vanadium, yttrium, and a lanthanide; and y is an integer from 3 to 6; x is 1 or 2; and

wherein the composition has a pH of 4.5 to 6.5.

The use of the pH range 4.5 to 6.5 may provide increased versatility in the choice of the metal ion, for example, when preparing the composition by binding biological material to the immobilized-metal support material. For example, the metal ion, Ga³⁺ effectively binds bacterial cells at a pH of 4.5 to 6.5, but may release cells at a pH of 7 to 9. A pH in the range of 4.5 to 6.5 can be conveniently provided using a 0.1 M 4-morpholineethanesulfonic acid (MES) buffer at a pH of about 5.5. For certain embodiments, including any one of the above compositions, the composition has a pH of 5 to 6.

In order to minimize interference with methods in which the compositions of the present invention may be used, appreciable levels of a salt may optionally not be included. Appreciable level(s) refers to a level greater than about 0.2 M, and more preferably a level greater than about 0.1 M. For certain embodiments, when a salt is present in the composition at an appreciable level, any salt included at an appreciable level in the composition is other than an inorganic salt or a one to four carbon atom-containing salt.

For certain embodiments, including any one of the above compositions, the plurality of —C(O)O⁻ or —P(O)(—OH)₂₋ₓ(—O⁻)ₓ groups is a plurality of —C(O)O⁻ groups.

The metal ion, M^{y+}, is chosen so that the metal ion can bind the phosphate portion of the polynucleotide sufficiently to bind the polynucleotide molecules present in a sample material. Moreover, the metal ion is also chosen to allow competitive binding with a metal-chelating reagent in a wash buffer to efficiently, preferably quantitatively, release or elute the polynucleotide molecules from the immobilized-metal support material at a low reagent concentration and under mild conditions. A low reagent concentration without the addition of any salt to increase the ionic strength can be about 0.1 M or less, 0.05 M or less, or 0.025 M or less. Mild conditions can include the low reagent concentration, a pH of about 7 to 10, a temperature of not more than about 95° C., preferably not more than about 65° C., or a combination thereof.

For certain embodiments, including any one of the above embodiments, M is selected from the group consisting of zirconium, gallium, iron, aluminum, scandium, titanium, vanadium, yttrium, and a lanthanide. A lanthanide includes any one of the lanthanide metals: lanthanum, cerium, praseodymium, promethium, samarium, europium, gadolinium, terbium, dysprosium, holmium, erbium, thulium, ytterbium, and lutetium. Lanthanum and cerium are preferred lanthanides. For certain of these embodiments, M is selected from the group consisting of zirconium, gallium, iron, aluminum, scandium, titanium, vanadium, lanthanum, and cerium. For certain of these embodiments, M is selected from the group consisting of zirconium, gallium, and iron. For certain of these embodiments, M is zirconium.

For certain embodiments, including any one of the above embodiments, y is 3 or 4.

For certain embodiments, including any one of the above embodiments, MY is Zr⁴⁺ or Ga³⁺. For certain of these embodiments, M^{y+} is Zr

In another embodiment, there is provided a method of separating and optionally assaying at least one double stranded polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, MY bound to the —C(O)O⁻ or —P(O)(—OH)₂₋ₓ(—O⁻)ₓ groups to provide a composition comprising a) the at least one double stranded polynucleotide bound to the immobilized-metal support material and b) a supernate comprising the sample material having a reduced amount of the at least one double stranded polynucleotide;

wherein M is selected from the group consisting of zirconium, gallium, iron, aluminum, scandium, titanium, vanadium, yttrium, and a lanthanide; y is an integer from 3 to 6; and x is 1 or 2.

In another embodiment, there is provided a method of separating and optionally assaying at least one polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, M^{y+}, bound to the —C(O)O⁻ or —P(O)(—OH)₂₋ₓ(—O⁻)ₓ groups, at a pH of 4.5 to 6.5, to provide a composition comprising a) the at least one polynucleotide bound to the immobilized-metal support material and b) a supernate comprising the sample material having a reduced amount of the at least one polynucleotide;

wherein M is selected from the group consisting of zirconium, gallium, iron, aluminum, scandium, titanium, vanadium, yttrium, and a lanthanide; y is an integer from 3 to 6; and x is 1 or 2; and

wherein the composition has a pH of 4.5 to 6.5.

For certain embodiments, including any one of the above methods, the composition has a pH of 5 to 6.

For certain embodiments, including any one of the above methods, any salt included at an appreciable level in the composition is other than an inorganic salt or a one to four carbon atom-containing salt.

The sample material is any material which may contain a polynucleotide. The sample material can be a raw sample material or a processed sample material. Raw sample materials include, for example, clinical samples or specimens (blood, tissue, etc.), food samples (foods, feeds, including pet food, beverages, raw materials for foods or feeds, etc.), environmental samples (water, soil, etc.), or the like. Processed sample materials include, for example, samples containing cells or viruses separated from a raw sample material, and samples containing polynucleotides isolated from cells, viruses, or derived from other sources. Some examples of sample material, such as clinical samples or specimens, include nasal, throat, sputum, blood, wound, groin, axilla, perineum, and fecal samples.

For certain embodiments, including any one of the above methods, the sample material includes a biological material containing a nucleic acid. For certain of these embodiments, the sample material includes a plurality of cells, viruses, or a combination thereof. For certain of these embodiments, the sample material includes a plurality of cells. Cells can be prokaryotic or eukaryotic cells, and can include mammalian and non-mammalian animal cells, plant cells, algae, including blue-green algae, fungi, bacteria, protozoa, yeast, and the like. For certain of these embodiments, the cells are bacterial cells, yeast cells, mold cells, or a combination thereof. For certain of these embodiments, the cells are bacterial cells.

For certain embodiments, including any one of the above embodiments wherein the sample material includes a plurality of cells, viruses, or a combination thereof, the method further comprises adding a lysis reagent to the sample material prior to contacting the sample material with the plurality of metal ions, M^{y+}, bound to the —C(O)O⁻ or —P(O)(—OH)₂₋ₓ(—O⁻)ₓ groups. For certain of these embodiments where the sample material contains at least one double stranded polynucleotide, the method further comprising lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide. Alternatively, for certain of these embodiments where the sample material contains at least one polynucleotide, the method further comprising lysing the cells, viruses, or a combination thereof to provide the composition comprising a) the at least one polynucleotide bound to the immobilized-metal support material and b) the supernate comprising the sample material having a reduced amount of the at least one polynucleotide.

Lysing can be carried out ezymatically, chemically, and/or mechanically. Enzymes used for lysis include, for example, lysostaphin, lysozyme, mutanolysin, or others. Chemical lysis can be carried out using a surfactant, alkali, heat, or other means. When alkali is used for lysis, a neutralization reagent may be used to neutralize the solution or mixture after lysis. Mechanical lysis can be accomplished by mixing or shearing using solid particles or microparticles such as beads or microbeads. Sonication may also be used for lysis. The lysis reagent can include a surfactant or detergent such as sodium dodecylsulfate (SDS), lithium laurylsulfate (LLS), TRITON series, TWEEN series, BRIJ series, NP series, CHAPS, N-methyl-N-(1-oxododecyl)glycine, sodium salt, or the like, buffered as needed; a chaotrope such as guanidium hydrochloride, guanidium thiacyanate, sodium iodide, or the like; a lysis enzyme such as lysozyme, lysostaphin, mutanolysin, proteinases, pronases, cellulases, or any of the other commercially available lysis enzymes; an alkaline lysis reagent; solid particles such as beads, or a combination thereof.

For certain embodiments, including any one of the above embodiments wherein the sample material includes a plurality of cells, viruses, or a combination thereof, alternatively, the sample material is contacted with a lysis reagent when contacting the sample material with the plurality of metal ions, M^{y+}, bound to the —C(O)O⁻ or —P(O)(—OH)₂₋ₓ(—O⁻)ₓ groups. In this alternative method, the number of steps can be reduced by simultaneously binding the plurality of cells, viruses, or a combination thereof to the plurality of metal ions, M^{y+}, bound to the —C(O)O⁻ or —P(O)(—OH)₂₋ₓ(—O⁻)ₓ groups, lysing the cells, viruses, or a combination thereof, and binding the polynucleotides from the cells, viruses, or a combination thereof. For certain of these embodiments where the sample material contains at least one double stranded polynucleotide, the method further comprises lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide. Alternatively, for certain of these embodiments where the sample material contains at least on polynucleotide, the method further comprises lysing the cells, viruses, or a combination thereof to provide the composition comprising a) the at least one polynucleotide bound to the immobilized-metal support material and b) the supernate comprising the sample material having a reduced amount of the at least one polynucleotide.

For certain embodiments, including any one of the above methods where the sample material including a plurality of cells, viruses, or a combination thereof is contacted with the plurality of metal ions, M^{y+}, bound to the —C(O)O⁻ or —P(O)(—OH)₂₋ₓ(—O⁻)ₓ groups, there is provided a) at least a portion of the plurality of cells, viruses, or a combination thereof bound to the immobilized-metal support material and b) a supernate comprising the sample material having a reduced number of cells, viruses, or a combination thereof. For certain of these embodiments, the method further comprises separating the supernate comprising the sample material having a reduced number of cells, viruses, or a combination thereof from the at least a portion of the plurality of cells, viruses, or a combination thereof bound to the immobilized-metal support material.

Separating the supernate from the immobilized-metal support material can be carried out, for example, by decanting, centrifuging, pipetting, and/or a combination of these methods. When the support material is comprised of magnetic particles, the immobilized-metal support material can be held in place at a wall of the chamber or container by applying a magnetic field. The supernate can then be removed by decanting, pipetting, or forcing the supernate out of the chamber or container using a pressure differential or a g-force.

For certain of these embodiments, the method further comprising washing the cells, viruses, or a combination thereof bound to the immobilized-metal support material. For certain of these embodiments, the method further comprises assaying the cells, viruses, or a combination thereof bound to the immobilized-metal support material. Alternatively, the method further comprises separating the cells, viruses, or a combination thereof from the immobilized-metal support material. For certain of these embodiments, the method further comprises assaying the cells, viruses, or a combination thereof. The assaying can be carried out using known assays such as colorimetric assays, immunoassays, or the like.

For certain embodiments, including any one of the above methods where at least a portion of the plurality of cells, viruses, or a combination thereof are bound to the immobilized-metal support material, except for the methods where adding a lysis reagent is included, the method further comprises adding a lysis reagent to the at least a portion of the plurality of cells, viruses, or a combination thereof bound to the immobilized-metal support material. For certain of these embodiments where the sample material contains at least one double stranded polynucleotide, the method further comprising lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide. Alternatively, for certain of these embodiments where the sample material contains at least on polynucleotide, the method further comprising lysing the cells, viruses, or a combination thereof to provide the composition comprising a) the at least one polynucleotide bound to the immobilized-metal support material and b) the supernate comprising the sample material having a reduced amount of the at least one polynucleotide.

For certain embodiments, including any one of the above embodiments which includes cells, viruses, or a combination thereof, the cells, viruses, or a combination thereof are cells. For certain of these embodiments, the cells are bacterial cells. The bacteria can be gram-positive or gram-negative. For certain of these embodiments where the bacterial cells are bound to the immobilized-metal support material, the bacterial cells are bound to the immobilized-metal support material in the presence of a binding buffer at a pH of 4.5 to 9. For certain of these embodiments, the pH is 4.5 to 6.5. In one example, the binding buffer is MES at about 0.1 M and at a pH of about 5.5. A non-ionic surfactant such as PLURONIC L64 (a polyoxyethylene-polyoxypropylene block copolymer available from BASF (Mt. Olive, N.J.) or TRITON X-100 (polyoxyethylene(10) isooctylphenyl ether available from Sigma-Aldrich, St. Louis, Mo.) can be included for improved flow and mixing. Surfactants may also reduce or prevent clumping of bacterial cells. Other buffers which can be similarly used include succinic acid, acetate, or citrate.

For certain embodiments, including any one of the above methods that includes providing the composition comprising a) the at least one double stranded polynucleotide bound to the immobilized-metal support material and b) the supernate comprising the sample material having a reduced amount of the at least one double stranded polynucleotide, the method further comprises separating a) the at least one double stranded polynucleotide bound to the immobilized-metal support material from b) the supernate comprising the sample material having a reduced amount of the at least one double stranded polynucleotide. For certain of these embodiments, the method further comprises washing the separated at least one double stranded polynucleotide bound to the immobilized-metal support material with an aqueous buffer solution at a pH of 4.5 to 9. For certain of these embodiments, the aqueous buffer solution is at a pH of 4.5 to 6.5.

Examples of wash buffers include MES buffer, Tris buffer, HEPES buffer, phosphate buffer, TAPS buffer, and DIPSO (3-(N,N-bis[2-hydroxyethyl]amino)-2-hydroxypropanesulfonic acid) buffer.

For certain embodiments, including any one of the above methods which includes the separated at least one double stranded polynucleotide bound to the immobilized-metal support material, the method further comprises amplifying the at least one double stranded polynucleotide bound to the immobilized-metal support material to provide a plurality of amplicons. Known amplification methods such as those described supra which are applicable to amplifying DNA can be used here, for example, PCR or TMA. Amplifying can include the presence of one or more enzymes, for example, a thermostable DNA polymerase for PCR, or an RNA polymerase and a reverse transciptase for TMA. The amplicons can be selected from the group consisting of amplicons bound to the immobilized-metal support material, unbound amplicons, and a combination thereof. Alternatively, the method further comprises releasing the at least one double stranded polynucleotide bound to the immobilized-metal support material from the immobilized-metal support material; and separating the at least one double stranded polynucleotide from the immobilized-metal support material. For certain of these embodiments, the method further comprises amplifying the at least one double stranded polynucleotide. A plurality of amplicons can thereby be provided. For certain of these embodiments, with the double stranded polynucleotide bound or separated, amplifying includes heating the double stranded polynucleotide to at least one temperature of about 37 to 100° C. For certain of these embodiments, amplifying includes heating the double stranded polynucleotide to a temperature of about 94 to 97° C. At this temperature the two strands of DNA separate, resulting in single-stranded DNA templates. Amplifying may further include heating at additional temperatures, for example, at a temperature of about 37 to 74° C. At these temperatures, the primers can anneal to the DNA templates, and the resulting annealed primers can be extended along the DNA template by the enzyme that is present. For certain of these embodiments, amplifying includes heating at a temperature of about 40 to 65° C., about 55 to 65° C., about 58 to 62° C., or about 60° C. Both the annealing and the extension can occur at these temperatures. However, an additional temperature may be used to optimize the temperature for the particular enzyme used. For example, an additional temperature of about 70 to 74° C. may be used for the extension. Known methods can be used to cycle through these temperatures or temperature ranges to facilitate amplifying the polynucleotide. Alternatively, for certain of these embodiments, with the double stranded polynucleotide bound or separated, amplifying includes heating the double stranded polynucleotide to a temperature of about 37 to 44° C., for example, about 42° C. At these temperatures, which can be held constant, enzymes such as RNA polymerase and reverse transcriptase can produce RNA amplicons, resulting in a high level of amplification. Optionally, prior to amplification, the double stranded polynucleotide can be heated to a higher temperature, such as about 55 to 100° C.

For certain embodiments, including any one of the above methods that includes providing the composition comprising a) the at least one polynucleotide bound to the immobilized-metal support material and b) the supernate comprising the sample material having a reduced amount of the at least one polynucleotide, the method further comprises separating a) the at least one polynucleotide bound to the immobilized-metal support material from b) the supernate comprising the sample material having a reduced amount of the at least one polynucleotide. For certain of these embodiments, the method further comprises washing the separated immobilized-metal support material (with bound polynucleotide) with an aqueous buffer solution at a pH of 4.5 to 9. For certain of these embodiments, the aqueous buffer solution is at a pH of 4.5 to 6.5.

For certain embodiments, including any one of the above methods which includes the separated at least one polynucleotide bound to the immobilized-metal support material, the method further comprises amplifying the at least one polynucleotide bound to the immobilized-metal support material to provide a plurality of amplicons. Known amplification methods such as those described supra, for example, PCR or TMA, can be used here. The amplicons can be selected from the group consisting of amplicons bound to the immobilized-metal support material, unbound amplicons, and a combination thereof. Alternatively, the method further comprises releasing the at least one polynucleotide bound to the immobilized-metal support material from the immobilized-metal support material; and separating the at least one polynucleotide from the immobilized-metal support material. For certain of these embodiments, the method further comprises amplifying the at least one polynucleotide. A plurality of amplicons can thereby be provided. For certain of these embodiments, with the polynucleotide bound or separated, amplifying includes heating the polynucleotide to at least one temperature of about 37 to 100° C. For certain of these embodiments, where the polynucleotide is double stranded, amplifying includes heating to a temperature of about 94 to 97° C. as described supra. Whether the polynucleotide is single or double stranded, amplifying may further include heating at additional temperatures, for example, at a temperature of about 37 to 74° C. At these temperatures, the primers can anneal to the polynucleotide templates, and the resulting annealed primers can be extended along the polynucleotide template by the enzyme that is present. For certain of these embodiments, amplifying includes heating at a temperature of about 40 to 65° C., about 55 to 65° C., about 58 to 62° C., or about 60° C. Both the annealing and the extension can occur at these temperatures. However, an additional temperature may be used to optimize the temperature for the particular enzyme used. For example, an additional temperature of about 70 to 74° C. may be used for the extension. Known methods can be used to cycle through these temperatures or temperature ranges to facilitate amplifying the polynucleotide. Alternatively, for certain of these embodiments, with the polynucleotide bound or separated, amplifying includes heating the polynucleotide to a temperature of about 37 to 44° C., for example, about 42° C. At these temperatures, which can be held constant, enzymes such as RNA polymerase and reverse transcriptase can produce RNA amplicons, resulting in a high level of amplification. Optionally, the polynucleotide can be heated to a temperature, such as about 55 to 100° C., for example, about 60° C., prior to amplification. For certain of these embodiments, the at least one polynucleotide is a single stranded polynucleotide.

For certain embodiments, including any one of the above methods which includes providing a plurality of amplicons by amplifying a polynucleotide or double stranded polynucleotide bound to the immobilized metal support material, the method further comprises separating the amplicons from the immobilized-metal support material. In the case where the amount of immobilized-metal support material is sufficient to bind a large proportion of the amplicons, the method can include releasing and separating the amplicons and optionally the at least one polynucleotide or double stranded polynucleotide bound to the immobilized-metal support material, from the immobilized-metal support material. For certain of these embodiments, releasing the amplicons and optionally the at least one polynucleotide or double stranded polynucleotide is carried out at a pH of 7 to 10.

Releasing or eluting amplicons and polynucleotides can be carried out using an elution reagent. Examples of a suitable elution reagent include TES buffer, DIPSO buffer, TEA buffer, Tris buffer, phosphate buffer, pyrophosphate buffer, HEPES buffer, POPSO buffer, tricine buffer, bicine buffer, TAPS buffer, ammonium hydroxide, and sodium hydroxide. For certain embodiments, including any one of the above embodiments which includes releasing the amplicons and/or the at least one polynucleotide or the at least one double stranded polynucleotide, the releasing is carried out with an elution reagent selected from the group consisting of a phosphate buffer, a tris(hydroxymethyl)aminomethane (Tris) buffer, and sodium hydroxide. For certain of these embodiments, the elution reagent is phosphate buffer or Tris-EDTA buffer.

For certain embodiments, including any one of the above methods which includes amplifying the at least one double stranded polynucleotide, the method further comprises detecting the at least one double stranded polynucleotide.

For certain embodiments, including any one of the above methods which includes amplifying the at least one polynucleotide, the method further comprises detecting the at least one polynucleotide.

Probes can be used for detecting amplification products (amplicons) by fluorescing, and thereby generating a detectable signal, the intensity of which is dependent upon the number of fluorescing probe molecules. Probe molecules can be comprised of an oligonucleotide with a fluorescing group and a quenching group. Probes can fluoresce when separation or decoupling of the quenching group and the fluorescing group occurs upon binding to an amplicon or upon nucleic acid amplifying enzyme cleavage of the probe bound to the amplicon. Alternatively, a probe bound to the amplicon can fluoresce upon exposure to light of an appropriate wavelength.

For certain embodiment, including any one of the above methods, the plurality of —C(O)O⁻ or —P(O)(—OH)₂₋ₓ(—O⁻)ₓ groups is a plurality of —C(O)O⁻ groups.

For certain embodiment, including any one of the above methods, M is selected from the group consisting of zirconium, gallium, and iron.

For certain embodiment, including any one of the above methods, y is 3 or 4.

For certain embodiment, including any one of the above methods, M^{y+} is Zr⁴⁺ or Ga³⁺.

For certain embodiment, including any one of the above methods, M^{y+} is Zr⁴⁺.

For certain embodiment, including any one of the above methods, the method is carried out within a microfluidic device.

In another embodiment, there is provided a device for processing sample material, the device having:

at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one second chamber separate from the first chamber and capable of receiving and containing the fluid, the immobilized-metal support material, or both from the at least one first chamber;

wherein M is selected from the group consisting of zirconium, gallium, iron, aluminum, scandium, titanium, vanadium, yttrium, and the lanthanides; y is an integer from 3 to 6; and x is 1 or 2.

The device for processing sample material can provide a location or locations and conditions for sample preparation, nucleic acid amplification, and/or detection. The sample material may be located in one or a plurality of chambers. The device may provide uniform and accurate temperature control of one or more of the chambers. The device may provide channels between chambers, for example, such that sample preparation may take place in one or more chambers, and nucleic acid amplification and detection may take place in one or more other chambers. For certain embodiments, including any one of the above embodiments which include the device for processing sample material, the device for processing sample material is a microfluidic device. Some examples of microfluidic devices are described in U.S. Publication Numbers 2002/0064885 (Bedingham et al.); US2002/0048533 (Bedingham et al.); US2002/0047003 (Bedingham et al.); and US2003/138779 (Parthasarathy et al.); U.S. Pat. Nos. 6,627,159; 6,720,187; 6,734,401; 6,814,935; 6,987,253; 7,026,168, and 7,164,107; and International Publication No. WO 2005/061084 A1 (Bedingham et al.).

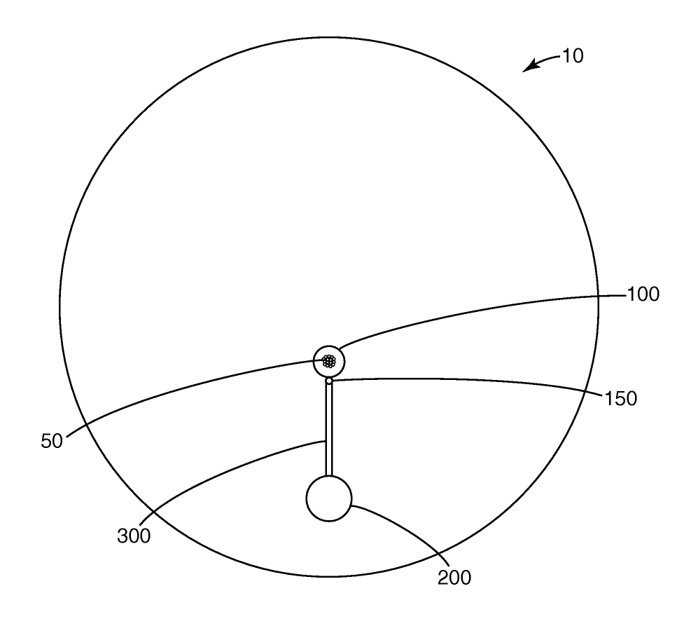

One illustrative device for processing sample material is the microfluidic device depicted in FIG. 1. The device 10 can be in the shape of a circular disc as illustrated in FIG. 1, although other shapes can be used. Preferred shapes are those that can be rotated. The device 10 of FIG. 1 comprises a first chamber 100 and a second chamber 200 which can be in fluid communication with the first chamber 100 via channel 300. The shape of chambers 100 and 200 can be circular as illustrated in FIG. 1, although other shapes, for example, oval, tear-drop, triangular, and many others can be used. FIG. 1 illustrates one combination of chamber 100 and chamber 200, but it is to be understood that a plurality of such combinations can be included in device 10 and may be desirable for simultaneously processing a plurality of samples.

The device 10 illustrated in FIG. 1 includes the immobilized-metal support material 50 in chamber 100. The immobilized-metal support material 50 can be a plurality of magnetic or non-magnetic particles such as microparticles (microspheres, microbeads, etc.), resin particles, or the like, illustrated in FIG. 1 as small circles. Alternatively, the immobilized-metal support material can be in the form of a filter, a frit, a film, a plurality of fibers, a glass slide, or the like, depending upon the substrate employed as described above. In another alternative, the immobilized-metal support material can be the interior walls of chamber 100.

Sample preparation such as binding cells or viruses, lysing, digesting debris from cells or viruses, polynucleotide binding, washing, and the like to be carried out in chamber 100 prior to moving material in chamber 100 through channel 300 and into chamber 200. After the polynucleotide has been separated from the sample material by binding to the immobilized metal support material, the immobilized metal support material can be moved to chamber 200, or the polynucleotide can be eluted from the immobilized metal support material and the resulting eluant moved to chamber 200. The channel 300 can provide a path for a fluid and/or the immobilized-metal support material in chamber 100 to move into chamber 200. This can be carried out, for example, by applying a sufficient g-force to the fluid and/or the immobilized-metal support material in the form of particles to force the material through channel 300 and into chamber 200. Alternatively, a pressure differential can be applied to channel 300, for example, by reducing the pressure in chamber 200, by increasing the pressure in chamber 100, or both, thereby causing material in chamber 100 to move through channel 300 and into chamber 200. Chamber 100 or channel 300 can be equipped with optional valve 150. Valve 150 can be fabricated to open by exposure to a sufficient g-force, by melting, by vaporizing, or the like. For example, the valve can be fabricated in the form of a septum in which an opening can be formed through laser ablation, focused optical heating, or similar means. Such valves are described, for example in U.S. Patent Application Publication Nos. 2005/0126312 A1 (Bedingham et al.) and 2005/0142571 A1 (Parthasarathy et al.).

Although not shown in FIG. 1, chambers 100 and 200 and channel 300 can be in fluid communication with other chambers, channels, reservoirs, and/or the like. These can be used to facilitate supplying or removing various reagents, sample material(s), or a component(s) of a sample material to or from chambers 100 or 200 as needed. For example, sample materials, lysis reagents, digestion reagents, wash buffers, binding buffers, elution buffers, and/or the like can be supplied to and/or removed from chamber 100, and primers, nucleotide triphosphates, amplifying enzymes, probes, buffers, and/or the like can be supplied to chamber 200. Individual reagents or combinations of reagents can be placed in different chambers, whether included in the device 10 or in any embodiment of the device described herein, to subsequently contact the reagents with the sample material or a component of the sample material as desired.

For certain embodiments, including any one of the above embodiments of the device for processing sample material, the at least one first chamber further contains a lysis reagent. The lysis reagent can include any one or any combination of lysis reagents described above.

For certain embodiments, including any one of the above embodiments of the device for processing sample material, a plurality of cells are bound to the immobilized-metal support material. For certain of these embodiments, the cells are bacterial cells.

For certain embodiments, including any one of the above embodiments of the device for processing sample material, at least one polynucleotide is bound to the immobilized-metal support material. For certain of these embodiments, the at least one polynucleotide is at least one double stranded polynucleotide.

For certain embodiments, including any one of the above embodiments of the device for processing sample material where at least one polynucleotide is bound to the immobilized-metal support material, the first chamber further contains a supernate having a pH of 4.5 to 6.5. For certain of these embodiments, the supernate has a pH of 5 to 6. For certain of these embodiments, any salt included at an appreciable level in the supernate is other than an inorganic salt or a one to four carbon atom-containing salt.

For certain embodiments, including any one of the above embodiments of the device for processing sample material where at least one double stranded polynucleotide is bound to the immobilized-metal support material, the first chamber further contains a supernate having a pH of 4.5 to 9. For certain of these embodiments, the supernate has a pH of 5.5 to 8.0.

For certain embodiments, including any one of the above embodiments of the device for processing sample material, the plurality of —C(O)O⁻ or —P(O)(—OH)₂₋ₓ(—O⁻)ₓ groups is a plurality of —C(O)O⁻ groups.

For certain embodiments, including any one of the above embodiments of the device for processing sample material, M is selected from the group consisting of zirconium, gallium, and iron.

For certain embodiments, including any one of the above embodiments of the device for processing sample material, y is 3 or 4.

For certain embodiments, including any one of the above embodiments of the device for processing sample material, M^{y+} is Zr⁴⁺ or Ga³⁺.

For certain embodiments, including any one of the above embodiments of the device for processing sample material, M^{y+} is Zr⁴⁺.

For certain embodiments, including any one of the above embodiments of the device for processing sample material the device is a microfluidic device.

For certain embodiments, including any one of the above embodiments of the device for processing sample material, at least one chamber of the device includes at least one additional reagent which can be used in at least one step of a nucleic acid manipulation technique. For certain of these embodiments, the at least one additional reagent can be used in a step of sample preparation, a step of nucleic acid amplification, and/or a step of detection in a process for detecting or assaying a nucleic acid. Sample preparation may include, for example, capturing a biological material containing a nucleic acid, washing a biological material containing a nucleic acid, lysing a biological material containing a nucleic acid, for example, cells or viruses, digesting cellular debris, isolating, capturing, or separating at least one polynucleotide or nucleic acid from a biological sample, and/or eluting a nucleic acid. Nucleic acid amplification may include, for example, producing a complementary polynucleotide of a polynucleotide or a portion of a polynucleotide in sufficient numbers for detection. Detection includes, for example, making an observation, such as detecting a fluorescence, which indicates the presence and/or amount of a polynucleotide. For certain of these embodiments, at least one chamber of the device includes at least one additional reagent selected from the group consisting of a nucleic acid amplifying enzyme, an oligonucleotide, a probe, nucleotide triphosphates, a buffer, a salt, a surfactant, a dye, a nucleic acid control, a reducing agent, Bovine Serum Albumin, dimethyl sulfoxide (DMSO), glycerol, ethylenediaminetetraacetic acid (EDTA), ethylene glycol-bis(2-aminoethylether)-N,N,N,N′-tetraacetic acid (EGTA), and a combination thereof. For certain of these embodiments, at least one chamber of the device includes at least one additional reagent selected from the group consisting of a nucleic acid amplifying enzyme, an oligonucleotide, a probe, nucleotide triphosphates, a buffer, and a salt.

“Nucleic acid amplifying enzyme” refers to an enzyme which can catalyze the production of a polynucleotide or a nucleic acid from an existing DNA or RNA template. For certain embodiments, the nucleic acid amplifying enzyme is an enzyme that can be used in a process for amplifying a nucleic acid or a portion of a nucleic acid. For certain embodiments, the nucleic acid amplifying enzyme is selected from the group consisting of a DNA and/or RNA polymerase and a reverse transcriptase. For certain embodiments, the DNA polymerase is selected from the group consisting of Taq DNA polymerase, Tfl DNA polymerase, Tth DNA polymerase, Tli DNA polymerase, and Pfu DNA polymerase. For certain of these embodiments, the reverse transcriptase is selected from the group consisting of AMV reverse transcriptase, M-MLV reverse transcriptase, and M-MLV reverse transcriptase, RNase H minus. Retroviral reverse transcriptase, such as M-MLV and AMV posses an RNA-directed DNA polymerase activity, a DNA directed polymerase activity, as well as an RNase H activity. For certain embodiments, the nucleic acid amplifying enzyme is a DNA polymerase or an RNA polymerase. For certain embodiments, the nucleic acid amplifying enzyme is Taq DNA polymerase. For certain embodiments, the nucleic acid amplifying enzyme is T7 RNA polymerase.

The “oligonucleotide” can be a primer, a terminating oligonucleotide, an extender oligonucleotide, or a promoter oligonucleotide. For certain embodiments, the oligonucleotide is a primer. Such oligonucleotides typically comprised of 15 to 30 nucleotide units, which determines the region (targeted sequence) of a nucleic acid to be amplified. Under appropriate conditions, the bases in the primer bind to complementary bases in the region of interest, and then the nucleic acid amplifying enzyme extends the primer as determined by the targeted sequence. A large number of primers are known and commercially available, and others can be designed and made using known methods.

Probes allow detection of amplification products (amplicons) by fluorescing, and thereby generating a detectable signal, the intensity of which is dependent upon the number of fluorescing probe molecules. Probe molecules can be comprised of an oligonucleotide and a fluorescing group coupled with a quenching group. Probes can fluoresce when separation or decoupling of the quenching group and the fluorescing group occurs upon binding to an amplicon or upon nucleic acid amplifying enzyme cleavage of the probe bound to the amplicon. Alternatively, a probe bound to the amplicon can fluoresce upon exposure to light of an appropriate wavelength. For certain embodiments, including any one of the above embodiments, the probe is selected from the group consisting of TAQMAN probes (Applied Biosystems, Foster City, Calif.), molecular beacons, SCORPIONS probes (Eurogentec Ltd., Hampshire, UK), SYBR GREEN (Invitrogen, Carlsbad, Calif.), FRET hybridization probes (Roche Applied Sciences, Indianapolis, Ind.), Quantitect probes (Qiagen, Valencia, Calif.), and molecular torches.

The nucleotide triphosphates (NTPs), including ribonucleotide triphosphates and deoxyribonucleotides triphosphates as required, are used by the nucleic acid amplifying enzyme in the production of a polynucleotide or a nucleic acid from an existing DNA or RNA template. For example, when amplifying a DNA, a dNTP (deoxyribonucleotide triphosphate) set is used, which typically includes dATP (2′-deoxyadenosine 5′-triphosphate), dCTP (2′-deoxycytodine 5′-triphosphate), dGTP (2′-deoxyguanosine 5′-triphosphate), and dTTP (2′-deoxythimidine 5′-triphosphate).

Buffers are used to regulate the pH of the reaction media. A wide variety of buffers are known and commercially available. For example, morpholine buffers, such as 2-(N-morpholino)ethanesulfonic acid (MES), can be suitable for providing an effective pH range of about 5.0 to 6.5, imidazole buffers can be suitable for providing an effective pH range of about 6.2 to 7.8, and tris(hydroxymethyl)aminomethane (TRIS) buffers and certain piperazine buffers such as N-(2-hydroxyethyl)piperazine-N'-(2-ethanesulfonic acid) (HEPES) can be suitable for providing an effective pH range of about 7.0 to 9.0. The buffer can affect the activity and fidelity of nucleic acid amplifying enzymes, such as polymerases. For certain embodiments, the buffer is selected from at least one buffer which can regulate the pH in the range of 7.5 to 8.5. For certain of these embodiments, the buffer is a TRIS-based buffer. For certain of these embodiments, the buffer is selected from the group consisting of at least one of TRIS-EDTA, TRIS buffered saline, TRIS acetate-EDTA, and TRIS borate-EDTA. Other materials can be included with these buffers, such as surfactants and detergents, for example, CHAPS or a surfactant described below. The buffers may be free of RNase and DNase.

Salts can affect the activity of nucleic acid amplifying enzymes. For example, free magnesium ions are necessary for certain polymerases, such as Taq DNA polymerase, to be active. In another example, in the presence of manganese ions, Tfl DNA polymerase and Tth DNA polymerase can catalyze the polymerization of nucleotides into DNA, using RNA as a template. In a further example, the presence of certain salts, such as potassium chloride, can increase the activity of certain polymerases such as Taq DNA polymerase. For certain embodiments, including any one of the above embodiments, the salt is selected from the group consisting of at least one of magnesium, manganese, zinc, sodium, and potassium salts. For certain of these embodiments, the salt is at least one of magnesium chloride, manganese chloride, zinc sulfate, zinc acetate, sodium chloride, and potassium chloride. For certain of these embodiments, the salt is magnesium chloride.

A surfactant can be included for lysing or de-clumping cells, improving mixing, enhancing fluid flow, for example, in a device, such as a microfluidic device. The surfactant can be non-ionic, such as a poly(ethylene oxide)-polypropylene oxide) copolymer available, for example, under the trade name PLURONIC, polyethylene glycol (PEG), polyoxyethylenesorbitan monolaurate available under the trade name TWEEN 20, 4-(1,1,3,3-tetramethylbutyl)phenyl-polyethylene glycol available under the trade name Triton X-100; anionic, such as lithium lauryl sulfate, N-lauroylsarcosine sodium salt, and sodium dodecyl sulfate; cationic, such as alkyl pyridinium and quaternary ammonium salts; zwitterionic, such as N—(C₁₀-C₁₆ alkyl)-N,N-dimethylglycine betaine (in the betaine family of surfactants); and/or a fluoro surfactant such as FLUORAD-FS 300 (3M, St. Paul, Minn.) and ZONYL (Dupont de Nemours Co., Wilmington, Del.).

A dye can be included in the reagent layer to impart a color or a fluorescence to the reagent layer or to a fluid which contacts the reagent layer. The color or fluorescence can provide visual evidence or a detectable light absorption or light emission evidencing that the reagent layer has been dissolved, dispersed, or suspended in the fluid which contacts the reagent layer. For certain embodiments, the dye is selected from the group consisting of fluorescent dyes, such as fluorescein, cyanine (which includes Cy3 and Cy5), Texas Red, ROX, FAM, JOE, SYBR Green, OliGreen, and HEX. In addition to these fluorescent dyes, ultraviolet/visible dyes, such as dichlorophenol, indophenol, saffranin, crystal violet, and commercially-available food coloring can also be used.

A nucleic acid control is a known amount of a nucleic acid or nucleic acid containing material dried-down with either the sample preparation or the amplification or detection reagents. This internal control can be used to monitor reagent integrity as well as inhibition from the sample material or specimen. Linearized plasmid DNA control is typically used as a nucleic acid internal control.

The reducing agent is a material capable of reducing disulfide bonds, for example in proteins which can be present in a sample material or specimen, and thereby reduce the viscosity and improve the flow and mixing characteristics of the sample material. For certain embodiments, the reducing agent preferably contains at least one thiol group. Examples of reducing agent include N-acetyl-L-cysteine, dithiothreitol, 2-mercaptoethanol, and 2-mercaptoethylamine.

Bovine Serum Albumin can be used to stabilize the enzyme during nucleic acid amplification; dimethyl sulfoxide (DMSO) can be used to inhibit the formation of secondary structures in the DNA template; glycerol can improve the amplification process, can be used as a preservative, and can stabilize enzymes such as polymerases; ethylenediaminetetraacectic acid (EDTA) and ethylene glycol-bis(2-aminoethylether)-N,N,N′N′-tetraacetic acid (EGTA) can be used as metal ion chelators and also to inactivate metal-binding enzymes (RNases) that may damage the reaction.

In another embodiment, there is provided a kit for separating at least one polynucleotide from a sample material, the kit comprising:

a device having at least one chamber capable of containing or channeling a fluid;

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and the lanthanides; y is an integer from 3 to 6; and x is 1 or 2; and

at least one reagent selected from the group consisting of a lysis reagent, a lysis buffer, a binding buffer, a wash buffer, and an elution buffer. For certain embodiments of this kit, the at least one chamber contains the immobilized-metal support material. For certain of these embodiments, the immobilized-metal support material substrate is selected from the group consisting of the interior walls of a column, a filter, a microplate, a microfilter plate, a microtiter plate, a frit, a pipette tip, a film, a plurality of microspheres, a plurality of fibers, and a glass slide.

In another embodiment, there is provided a kit for separating and optionally assaying at least one polynucleotide from a sample material, the kit comprising any one of the above embodiments of the device for processing sample material having:

at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one second chamber separate from the first chamber and capable of receiving and containing the fluid, the immobilized-metal support material, or both from the at least one first chamber;

wherein M is selected from the group consisting of zirconium, gallium, iron, aluminum, scandium, titanium, vanadium, yttrium, and the lanthanides; y is an integer from 3 to 6; and x is 1 or 2. For certain of these embodiments, the kit further comprises a reagent selected from the group consisting of a lysis reagent, a lysis buffer, a binding buffer, a wash buffer, an elution buffer, and a combination thereof. For certain of these embodiments, the at least one first chamber contains at least one reagent selected from the group consisting of a lysis reagent, a lysis buffer, a binding buffer, a wash buffer, an elution buffer, and a combination thereof. For certain of these embodiments, the at least one polynucleotide is at least one double stranded polynucleotide.

For certain embodiments, including any one of the above composition, method, device, or kit embodiments, the immobilized-metal support material substrate is a plurality of microspheres. For certain of these embodiments, the microspheres are magnetic. For certain of these embodiments, the microspheres have a diameter of 0.1 to 10 microns (μ).

For certain embodiments, including any one of the above method, device, or kit embodiments which includes a sample material, the sample material is selected from the group consisting of a food sample, nasal sample, throat sample, sputum sample, blood sample, wound sample, groin sample, axilla sample, perineum sample, and fecal sample. For certain embodiments the sample material is a nasal sample, a fecal sample, or a blood sample. For certain embodiments, the sample material is a fecal sample. For certain embodiments, the sample material is a blood sample.

In another embodiment, there is provided a microorganism binding composition comprising: an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and a plurality of microorganisms, selected from the group consisting of bacterial cells, yeast cells, mold cells, viruses, and a combination thereof, non-specifically bound to the immobilized-metal support material; wherein M is selected from the group consisting of zirconium, gallium, iron, aluminum, scandium, titanium, vanadium, yttrium, and a lanthanide; y is an integer from 3 to 6; and x is 1 or 2.

In another embodiment, there is provided method of isolating microorganisms comprising: providing a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; providing a sample suspected of having a plurality of microorganisms selected from the group consisting of bacterial cells, yeast cells, mold cells, viruses, and a combination thereof; and contacting the composition with the sample; wherein at least a portion of the plurality of microorganisms from the sample become non-specifically bound to the immobilized-metal support material; wherein M is selected from the group consisting of zirconium, gallium, iron, aluminum, scandium, titanium, vanadium, yttrium, and a lanthanide; y is an integer from 3 to 6; and x is 1 or 2.

For certain embodiments, including the above method of isolating microorganisms, the method further comprises separating the immobilized-metal support material from the remainder of the sample after the at least a portion of the plurality of microorganism from the sample become non-specifically bound to the immobilized-metal support material. For certain of these embodiments, the method further comprises detecting the at least a portion of the plurality of microorganisms. For certain of these embodiments, the detecting is carried out by a detection method selected from the group consisting of adenosine triphosphate (ATP) detection by bioluminescence, polydiacetylene (PDA) colorimetric detection, nucleic acid detection, immunological detection, growth based detection, visual detection by microscopy, magnetic resistance, and surface acoustic wave detection.

ATP detection can be used as a nonspecific indicator of microorganism load. After separating the solid support with non-specifically bound microorganisms from the remainder of the sample (which may contain interfering components such as extra-cellular ATP), the microorganisms are lysed and contacted with luciferin and luciferase. The resulting bioluminescence, which is of an intensity proportional to the number of captured microorganisms, is then measured, for example, using a luminometer.

PDA colorimetric detection can be used to detect specific microorganism or a spectrum of microorganisms by contacting a colorimetric sensor with the microorganism. The colorimetric sensor comprises a receptor and a polymerized composition which includes a diacetylene compound or a polydiacetylene. When microorganisms are bound by the receptor, resulting conformational changes to the sensor cause a measurable color change. The color change can be measured, for example, visually or using a colorimeter. Indirect detection of microorganisms using probes which can bind to the receptor may also be used. PDA colorimetric detection using such colorimetric sensors is known and described, for example, in U.S. Patent Application Publication No. 2006/0134796A1, International Publication Nos. WO 2004/057331A1 and WO 2007/016633A1, and in Assignee's co-pending U.S. Patent Application Ser. No. 60/989,298.

Methods for detecting nucleic acids, including DNA and RNA, often include amplifying or hybridizing the nucleic acids as described above after the captured microorganisms are lysed to make the cellular nucleic acids available for detection.

Immunological detection includes detection of a biological molecule, such as a protein, proteoglycan, or other material with antigenic activity, acting as a marker on the surface of bacteria. Detection of the antigenic material is typically by an antibody, a polypeptide selected from a process such as phage display, or an aptamer from a screening process. Immunological detection methods are known, examples of which include immunoprecipitation and enzyme-linked immunosorbent assays (ELISA). Antibody binding can be detected in several ways, including by labeling either the primary or the secondary antibody with a fluorescent dye, quantum dot, or an enzyme that can produce chemiluminescence or a color change. Plate readers and lateral flow devices have been used for detecting and quantifying the binding event. Growth based detection methods are well known and generally include plating the microorganisms, culturing the microorganisms to increase the number of microorganisms under specific conditions, and enumerating the microorganisms. PETRIFILM Aerobic Count Plates (3M Company, St. Paul, Minn.) can be used for this purpose.

Magnetic resistance detection is carried out by detection of a magnetic field generated by magnetic particles.

Surface acoustic wave detection, described, for example, in International Publication No. WO 2005/071416, is also known for detecting microorganisms. For example, a bulk acoustic wave-impedance sensor has been used for detecting the growth and numbers of microorganisms on the surface of a solid medium. The concentration range of the microorganisms that can be detected by this method was 3.4×10² to 6.7×10⁶ cells/ml. See Le Deng et al., J. Microbiological Methods, Vol. 26, Iss. 10-2, 197-203 (1997).

For certain embodiment, including the above microorganism binding compositions and methods of isolating microorganisms, M is selected from the group consisting of zirconium, gallium, and iron.

For certain embodiment, including the above microorganism binding compositions and methods of isolating microorganisms, y is 3 or 4.

For certain embodiment, including the above microorganism binding compositions and methods of isolating microorganisms, M^{y+} is Zr⁴⁺, Ga³⁺, or Fe³⁺ For certain of these embodiments, M^{y+} is Zr⁴⁺ or Ga³⁺ For certain of these embodiments, M^{y+} is Zr⁴⁺.

For certain embodiment, including the above microorganism binding compositions and methods of isolating microorganisms, the plurality of —C(O)O⁻ or —P(O)(—OH)₂₋ₓ(—O⁻)ₓ groups is a plurality of —C(O)O⁻ groups.

For certain embodiment, including the above microorganism binding compositions and methods of isolating microorganisms, the plurality of microorganisms includes two or more different types of bacteria, yeast, mold, or a combination thereof. For certain of these embodiments, the plurality of microorganisms includes two or more different types of bacteria.

For certain embodiment, including the above microorganism binding compositions and methods of isolating microorganisms, the microorganisms are selected from the group consisting of Bacillus, Bordetella, Borrelia, Campylobacter, Clostridium, Cornyebacteria, Enterobacter, Enterococcus, Escherichia, Helicobacter, Legionella, Listeria, Mycobacterium, Neisseria, Pseudomonas, Salmonella, Shigella, Staphylococcus, Streptococcus, Vibrio, Yersinia, Candida, Penicillium, Aspergillus, Cladosporium, Fusarium, and a combination thereof. In referring to above embodiments which include only bacteria, Candida, Penicillium, Aspergillus, Cladosporium, and Fusarium are not included.

For certain embodiment, including the above microorganism binding compositions and methods of isolating microorganisms, the microorganisms include Salmonella, E. coli, Campylobacter, Listeria, or a combination thereof.

For certain embodiment, including the above microorganism binding compositions and methods of isolating microorganisms, the substrate of the immobilized-metal support material is selected from the group consisting of a bead, a gel, a film, a sheet, a membrane, a particle, a fiber, a filter, a plate, a strip, a tube, a column, a well, a wall of a container, a capillary, a pipette tip, and a combination thereof. For certain of these embodiments, the substrate is magnetic particles. For certain of these embodiments, the magnetic particles have a diameter of about 0.02 to about 5 microns.

For certain embodiment, including the above microorganism binding compositions and methods of isolating microorganisms, the pH of the composition is 4.5 to 6.5. Microorganisms have been found to bind efficiently to the immobilized-metal support material in this pH range. For certain embodiments, the pH is preferably 5 to 6 or about 5.5.

For certain detection methods, it may be preferred to carry out the detection in the absence of the support material. PDA sensors, for example, can be strongly affected by the presence of magnetic particles. For certain embodiment, including the above methods of isolating microorganisms, the method further comprises releasing the microorganisms from the immobilized-metal support material by raising the pH to 8 to 10, and in some embodiments to about 9.

When M is zirconium, it has been found the effective microorganism binding can be carried out over a broader range of pH, for example, a range of about 4.5 to about 9.

Typically, zirconum is more effective at higher pH values than other choices of metal ions. For certain embodiment, including the above microorganism binding compositions and methods of isolating microorganisms, M is zirconium, and the pH of the composition is 4.5 to 9.

For certain embodiment, including the above microorganism binding compositions and methods of isolating microorganisms, the sample is selected from the group consisting of a clinical sample, a food sample, and an environmental sample. These samples may be a raw sample or a previously processed sample. For certain of these embodiments, the sample is a food sample.

### LIST OF EMBODIMENTS

The following is a listing of some of the embodiments described above, where “emb” means “embodiment” and “embs” means “embodiments”.

1. A composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one double stranded polynucleotide bound to at least one of the metal ions, M^{y+};

wherein M is selected from the group consisting of zirconium, gallium, iron, aluminum, scandium, titanium, vanadium, yttrium, and a lanthanide; y is an integer from 3 to 6; and x is 1 or 2.

2. A composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one polynucleotide bound to at least one of the metal ions, M^{y+};

wherein M is selected from the group consisting of zirconium, gallium, iron, aluminum, scandium, titanium, vanadium, yttrium, and a lanthanide; y is an integer from 3 to 6; and x is 1 or 2; and

wherein the composition has a pH of 4.5 to 6.5.

3. The composition of emb 2, wherein any salt included at an appreciable level in the composition is other than an inorganic salt or a one to four carbon atom-containing salt.
4. The composition of emb 2 or emb 3 wherein the composition has a pH of 5 to 6.
5. The composition of any one of embs 1 through 4, wherein the plurality of —C(O)O⁻ or —P(O)(—OH)₂₋ₓ(—O⁻)ₓ groups is a plurality of —C(O)O⁻ groups.
6. The composition of any one of embs 1 through 5, wherein M is selected from the group consisting of zirconium, gallium, and iron.
7. The composition of any one of embs 1 through 6, wherein y is 3 or 4.
8. The composition of any one of embs 1 through 7, wherein M^{y+} is Zr⁴⁺ or Ga³⁺.
9. A method of separating and optionally assaying at least one double stranded polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, M^{y+}, bound to the —C(O)O⁻ or —P(O)(—OH)₂₋ₓ(—O⁻)ₓ groups to provide a composition comprising a) the at least one double stranded polynucleotide bound to the immobilized-metal support material and b) a supernate comprising the sample material having a reduced amount of the at least one double stranded polynucleotide;

wherein M is selected from the group consisting of zirconium, gallium, iron, aluminum, scandium, titanium, vanadium, yttrium, and a lanthanide; y is an integer from 3 to 6; and x is 1 or 2.

10. A method of separating and optionally assaying at least one polynucleotide from a sample material comprising:

providing an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

contacting the sample material with the plurality of metal ions, M^{y+}, bound to the —C(O)O⁻ or —P(O)(—OH)₂₋ₓ(—O⁻)ₓ groups, at a pH of 4.5 to 6.5, to provide a composition comprising a) the at least one polynucleotide bound to the immobilized-metal support material and b) a supernate comprising the sample material having a reduced amount of the at least one polynucleotide;

wherein M is selected from the group consisting of zirconium, gallium, iron, aluminum, scandium, titanium, vanadium, yttrium, and a lanthanide; y is an integer from 3 to 6; and x is 1 or 2; and

wherein the composition has a pH of 4.5 to 6.5.

11. The method of emb 10, wherein any salt included at an appreciable level in the composition is other than an inorganic salt or a one to four carbon atom-containing salt.
12. The method of emb 10 or emb 11 wherein the composition has a pH of 5 to 6.
13. The method of any one of embs 9 through 12, wherein the sample material includes a biological material containing a nucleic acid.
14. The method of emb 13, wherein the sample material includes a plurality of cells, viruses, or a combination thereof.
15. The method of emb 14, further comprising adding a lysis reagent to the sample material prior to contacting the sample material with the plurality of metal ions, M^{y+}, bound to the —C(O)O⁻ or —P(O)(—OH)₂₋ₓ(—O⁻)ₓ groups.
16. The method of emb 14, wherein the sample material is contacted with a lysis reagent when contacting the sample material with the plurality of metal ions, M^{y+}, bound to the —C(O)O⁻ or —P(O)(—OH)₂₋ₓ(—O⁻)ₓ groups.
17. The method of emb 14, wherein contacting the sample material with the plurality of metal ions, M^{y+}, bound to the —C(O)O⁻ or —P(O)(—OH)₂₋ₓ(—O⁻)ₓ groups provides a) at least a portion of the plurality of cells, viruses, or a combination thereof bound to the immobilized-metal support material and b) a supernate comprising the sample material having a reduced number of cells, viruses, or a combination thereof.
18. The method of emb 17, further comprising separating the supernate comprising the sample material having a reduced number of cells, viruses, or a combination thereof from the at least a portion of the plurality of cells, viruses, or a combination thereof bound to the immobilized-metal support material.
19. The method of emb 18, further comprising washing the cells, viruses, or a combination thereof bound to the immobilized-metal support material.
20. The method of emb 19, further comprising assaying the cells, viruses, or a combination thereof bound to the immobilized-metal support material.
21. The method of emb 19, further comprising separating the cells, viruses, or a combination thereof from the immobilized-metal support material.
22. The method of emb 21, further comprising assaying the cells, viruses, or a combination thereof.
23. The method of emb 17 or emb 18, further comprising adding a lysis reagent to the at least a portion of the plurality of cells, viruses, or a combination thereof bound to the immobilized-metal support material.
24. The method of emb 16 or emb 23, each as dependent on emb 9, further comprising lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide.
25. The method of emb 16 or emb 23, each as dependent on any one of emb 10, 11, and 12, further comprising lysing the cells, viruses, or a combination thereof to provide the composition comprising a) the at least one polynucleotide bound to the immobilized-metal support material and b) the supernate comprising the sample material having a reduced amount of the at least one polynucleotide.
26. The method of any one of embs 14 through 25, wherein the cells, viruses, or a combination thereof are cells.
27. The method of emb 26, wherein the cells are bacterial cells.
28. The method of emb 27 as dependent on emb 17, wherein the bacterial cells are bound to the immobilized-metal support material in the presence of a binding buffer at a pH of 4.5 to 9.
29. The method of emb 28, wherein the pH is 4.5 to 6.5.
30. The method of any one of embs 9, 24, and 26 and 27 as dependent on emb 24, further comprising separating a) the at least one double stranded polynucleotide bound to the immobilized-metal support material from b) the supernate comprising the sample material having a reduced amount of the at least one double stranded polynucleotide.
31. The method of any one of embs 10, 11, 12, 25, and 26 and 27 as dependent on emb 19, further comprising separating a) the at least one polynucleotide bound to the immobilized-metal support material from b) the supernate comprising the sample material having a reduced amount of the at least one polynucleotide.
32. The method of emb 30, further comprising washing the separated at least one double stranded polynucleotide bound to the immobilized-metal support material with an aqueous buffer solution at a pH of 4.5 to 9.
33. The method of emb 31, further comprising washing the separated at least one polynucleotide bound to the immobilized-metal support material with an aqueous buffer solution at a pH of 4.5 to 6.5.
34. The method of emb 30 or emb 32, further comprising amplifying the at least one double stranded polynucleotide bound to the immobilized-metal support material to provide a plurality of amplicons.
35. The method of emb 34, wherein amplifying includes heating the double stranded polynucleotide to a temperature of about 94 to 97° C.
36. The method of emb 34 or emb 35, wherein amplifying includes heating the double stranded polynucleotide to a temperature of about 60° C.
37. The method of emb 34, wherein amplifying includes heating the double stranded polynucleotide to a temperature of about 37 to 44° C.
38. The method of emb 37, wherein the double stranded polynucleotide is heated to a temperature of about 60° C. prior to amplification.
39. The method of any one of embs 34 through 38, further comprising separating the amplicons from the immobilized-metal support material.
40. The method of emb 31 or emb 33, further comprising amplifying the at least one polynucleotide bound to the immobilized-metal support material to provide a plurality of amplicons.
41. The method of emb 40, wherein amplifying includes heating the at least one polynucleotide to a temperature of about 94 to 97° C.
42. The method of emb 40, wherein the at least one polynucleotide is a single stranded polynucleotide.
43. The method of emb 41 or emb 42, wherein amplifying includes heating the at least one polynucleotide to a temperature of about 60° C.
44. The method of emb 40 or emb 42, wherein amplifying includes heating the at least one polynucleotide to a temperature of about 37 to 44° C.
45. The method of emb 44, wherein the at least one polynucleotide is heated to a temperature of about 60° C. prior to amplification.
46. The method of any one of embs 40 through 45, further comprising separating the amplicons from the immobilized-metal support material.
47. The method of emb 30 or emb 32, further comprising releasing the at least one double stranded polynucleotide bound to the immobilized-metal support material from the immobilized-metal support material; and

separating the at least one double stranded polynucleotide from the immobilized-metal support material.

48. The method of emb 47, further comprising amplifying the at least one double stranded polynucleotide.
49. The method of emb 48, wherein amplifying includes heating the double stranded polynucleotide to a temperature of about 94 to 97° C.
50. The method of emb 49, wherein amplifying includes heating the double stranded polynucleotide to a temperature of about 60° C.
51. The method of emb 48, wherein amplifying includes heating the double stranded polynucleotide to a temperature of about 37 to 44° C.
52. The method of emb 51, wherein the double stranded polynucleotide is heated to a temperature of about 60° C. prior to amplification.
53. The method of any one of embs 39, and 48 through 52, further comprising detecting the at least one double stranded polynucleotide.
54. The method of emb 31 or emb 33, further comprising releasing the at least one polynucleotide bound to the immobilized-metal support material from the immobilized-metal support material; and

separating the at least one polynucleotide from the immobilized-metal support material.

55. The method of emb 54, wherein releasing the at least one polynucleotide bound to the immobilized-metal support material is carried out at a pH of 7 to 10.
56. The method of emb 54 or emb 55, wherein releasing the at least one polynucleotide bound to the immobilized-metal support material is carried out with an elution reagent selected from the group consisting of a phosphate buffer, a tris(hydroxymethyl)aminomethane buffer, and sodium hydroxide.
57. The method of any one of embs 54, 55, and 56, further comprising amplifying the at least one polynucleotide.
58. The method of emb 57, wherein amplifying includes heating the at least one polynucleotide to a temperature of about 94 to 97° C.
59. The method of emb 57, wherein the at least one polynucleotide is a single stranded polynucleotide.
60. The method of emb 58 or emb 59, wherein amplifying includes heating the at least one polynucleotide to a temperature of about 60° C.
61. The method of emb 57 or emb 59, wherein amplifying includes heating the at least one polynucleotide to a temperature of about 37 to 44° C.
62. The method of emb 61, wherein the at least one polynucleotide is heated to a temperature of about 60° C. prior to amplification.
63. The method of any one of embs 46, and 57 through 62, further comprising detecting the at least one polynucleotide.
64. The method of any one of embs 9 through 63, wherein the plurality of —C(O)O⁻ or —P(O)(—OH)₂₋ₓ(—O⁻)ₓ groups is a plurality of —C(O)O⁻ groups.
65. The method of any one of embs 9 through 64, wherein M is selected from the group consisting of zirconium, gallium, and iron.
66. The method of any one of embs 9 through 65, wherein y is 3 or 4.
67. The method of any one of embs 9 through 66, wherein M^{y+} is Zr⁴⁺ or Ga³⁺.
68. The method of any one of embs 9 through 67, wherein M^{y+} is Zr⁴⁺.
69. The method of any one of embs 9 through 68, wherein the method is carried out within a microfluidic device.
70. A device for processing sample material, the device having:

at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

at least one second chamber separate from the first chamber and capable of receiving and containing the fluid, the immobilized-metal support material, or both from the at least one first chamber;

wherein M is selected from the group consisting of zirconium, gallium, iron, aluminum, scandium, titanium, vanadium, yttrium, and the lanthanides; y is an integer from 3 to 6; and x is 1 or 2.

71. The device of emb 70, wherein the at least one first chamber further contains a lysis reagent.
72. The device of emb 70 or emb 71, wherein a plurality of cells are bound to the immobilized-metal support material.
73. The device of any one of embs 70 through 72, wherein at least one polynucleotide is bound to the immobilized-metal support material.
74. The device of emb 73, wherein the at least one polynucleotide is at least one double stranded polynucleotide.
75. The device of emb 73, wherein the first chamber further contains a supernate having a pH of 4.5 to 6.5.
76. The device of emb 75, wherein the supernate has a pH of 5 to 6.
77. The device of emb 74, wherein the first chamber further contains a supernate having a pH of 4.5 to 9.
78. The device of emb 77, wherein the supernate has a pH of 5.5 to 8.0.
79. The device of emb 75 or emb 76, wherein any salt included at an appreciable level in the supernate is other than an inorganic salt or a one to four carbon atom-containing salt.

80. The device of any one of embs 70 through 79, wherein the plurality of —C(O)O⁻ or —P(O)(—OH)₂₋ₓ(—O⁻)ₓ groups is a plurality of —C(O)O⁻ groups.

81. The device of any one of embs 70 through 80, wherein M is selected from the group consisting of zirconium, gallium, and iron.
82. The device of any one of embs 70 through 81, wherein y is 3 or 4.
83. The device of any one of embs 70 through 82, wherein M^{y+} is Zr⁴⁺ or Ga³⁺.
84. The device of any one of embs 70 through 83, wherein M^{y+} is Zr⁴⁺.
85. The device of any one of embs 70 through 84 wherein the device is a microfluidic device.
86. A kit for separating at least one polynucleotide from a sample material, the kit comprising:

a device having at least one chamber capable of containing or channeling a fluid;

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and the lanthanides; y is an integer from 3 to 6; and x is 1 or 2; and

at least one reagent selected from the group consisting of a lysis reagent, a lysis buffer, a binding buffer, a wash buffer, and an elution buffer.

87. The kit of emb 86, wherein the at least one chamber contains the immobilized-metal support material.
88. The kit of emb 86 or emb 87, wherein the at least one chamber is a column.
89. The kit of emb 86 or emb 87, wherein the at least one chamber is in a microfluidic device.

90. The kit of emb 86 or emb 87, wherein the immobilized-metal support material substrate is selected from the group consisting of the interior walls of a column, a filter, a microplate, a microfilter plate, a microtiter plate, a frit, a pipette tip, a film, a plurality of microspheres, a plurality of fibers, and a glass slide.

91. A kit for separating and optionally assaying at least one polynucleotide from a sample material, the kit comprising the device of any one of embs 70 through 85.
92. The kit of emb 91, further comprising a reagent selected from the group consisting of a lysis reagent, a lysis buffer, a binding buffer, a wash buffer, an elution buffer, and a combination thereof.

93. The kit of emb 92 wherein the at least one first chamber contains at least one reagent selected from the group consisting of a lysis reagent, a lysis buffer, a binding buffer, a wash buffer, an elution buffer, and a combination thereof.

94. The kit of any one of embs 86 through 93, wherein the at least one polynucleotide is at least one double stranded polynucleotide.
95. The composition of any one of embs 1 through 8, or the method of any one of embs 9 through 69, or the device of any one of embs 70 through 85, or the kit of any one of embs 86 through 94, wherein the immobilized-metal support material substrate is a plurality of microparticles.
96. The composition of emb 95, or the method of emb 95, or the device of emb 95, or the kit of emb 95, wherein the microparticles are magnetic.
97. The composition of any one of embs 95 and 96, or the method of any one of embs 95 and 96, or the device of any one of embs 95 and 96, or the kit of any one of embs 95 and 96, wherein the microparticles have a diameter of 0.1 to 10 microns.
98. The method of any one of embs 9 through 69 and 95 through 97, or the device of any one of embs 70 through 85 and 95 through 97, or the kit of any one of embs 86 through 94 and 95 through 97, wherein the sample material is selected from the group consisting of a food sample, nasal sample, throat sample, sputum sample, blood sample, wound sample, groin sample, axilla sample, perineum sample, and fecal sample.
99. A composition comprising:

an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and

a plurality of microorganisms, selected from the group consisting of bacterial cells, yeast cells, mold cells, viruses, and a combination thereof, non-specifically bound to the immobilized-metal support material;

wherein M is selected from the group consisting of zirconium, gallium, iron, aluminum, scandium, titanium, vanadium, yttrium, and a lanthanide; y is an integer from 3 to 6; and x is 1 or 2.

100. A method of isolating microorganisms comprising:

providing a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups;

providing a sample suspected of having a plurality of microorganisms selected from the group consisting of bacterial cells, yeast cells, mold cells, viruses, and a combination thereof; and

contacting the composition with the sample; wherein at least a portion of the plurality of microorganisms from the sample become non-specifically bound to the immobilized-metal support material;

wherein M is selected from the group consisting of zirconium, gallium, iron, aluminum, scandium, titanium, vanadium, yttrium, and a lanthanide; y is an integer from 3 to 6; and x is 1 or 2.

101. The method of emb 100, further comprising separating the immobilized-metal support material from the remainder of the sample after the at least a portion of the plurality of microorganism from the sample become non-specifically bound to the immobilized-metal support material.
102. The method of emb 101, further comprising detecting the at least a portion of the plurality of microorganisms.
103. The method of emb 102, wherein the detecting is carried out by a detection method selected from the group consisting of adenosine triphosphate (ATP) detection by bioluminescence, polydiacetylene (PDA) colorimetric detection, nucleic acid detection, immunological detection, growth based detection, visual detection by microscopy, magnetic resistance and surface acoustic wave detection.
104. The composition of emb 99 or the method of any one of embs 100 through 103,

wherein M is selected from the group consisting of zirconium, gallium, and iron.

105. The composition of emb 99 or emb 104 or the method of any one of embs 100 through 104, wherein y is 3 or 4.
106. The composition of any one of embs 99, 104, or 105 or the method of any one of embs 100 through 105, wherein M^{y+} is Zr⁴⁺, Ga³⁺, or Fe³⁺.
107. The composition of any one of embs 99, and 104 through 106 or the method of any one of embs 100 through 106, wherein the plurality of —C(O)O⁻ or —P(O)(—OH)₂₋ₓ(—O⁻)ₓ groups is a plurality of —C(O)O⁻ groups.
108. The composition of any one of embs 99 and 104 through 107 or the method of any one of embs 100 through 107, wherein the plurality of microorganisms includes two or more different types of bacteria, yeast, mold, or a combination thereof.
109. The composition of any one of embs 99 and 104 through 108 or the method of any one of embs 100 through 108, wherein the microorganisms are selected from the group consisting of Bacillus, Bordetella, Borrelia, Campylobacter, Clostridium, Cornyebacteria, Enterobacter, Enterococcus, Escherichia, Helicobacter, Legionella, Listeria, Mycobacterium, Neisseria, Pseudomonas, Salmonella, Shigella, Staphylococcus, Streptococcus, Vibrio, Yersinia, Candida, Penicillium, Aspergillus, Cladosporium, Fusarium, and a combination thereof.
110. The composition of emb 109 or the method of emb 109, wherein the microorganisms include Salmonella, E. coli, Campylobacter, Listeria, or a combination thereof.
111. The composition of any one of embs 99 and 104 through 110 or the method of any one of embs 100 through 110, wherein the substrate is selected from the group consisting of a bead, a gel, a film, a sheet, a membrane, a particle, a fiber, a filter, a plate, a strip, a tube, a column, a well, a wall of a container, a capillary, a pipette tip, and a combination thereof.
112. The composition of emb 111 or the method of emb 111, wherein the substrate is magnetic particles.
113. The composition of emb 112 or the method of emb 112, wherein the magnetic particles have a diameter of about 0.02 to about 5 microns.
114. The composition of any one of embs 99 and 104 through 113 or the method of any one of embs 100 though 113, wherein the pH of the composition is 4.5 to 6.5.
115. The method of any one of embs 100 though 114, further comprising releasing the microorganisms from the immobilized-metal support material by raising the pH to 8 to 10.
116. The composition or any one of embs 99 and 104 through 113 or the method of any one of embs 100 through 113, wherein M is zirconium, and the pH of the composition is 4.5 to 9.
117. The method of any one of embs 100 through 116, wherein the sample is selected from the group consisting of a clinical sample, a food sample, and an environmental sample.

Objects and advantages of this invention are further illustrated by the following examples, but the particular materials and amounts thereof recited in these examples, as well as other conditions and details, should not be construed to unduly limit this invention.

### EXAMPLES

### Example 1

### Preparation of Metal-Ion Mediated Magnetic Microparticles

Metal-ion mediated magnetic microparticles, for use as an immobilized-metal support material, were prepared from magnetic particles with surface carboxylic acid groups and with a diameter of about 1μ (DYNABEADS MYONE Carboxylic Acid from Invitrogen, Carlsbad, Calif., or SERA-MAG Magnetic Particles from Thermo Scientific (known as Seradyn, Indianapolis, Ind.). The carboxylated magnetic microparticles were placed in a tube and washed by attracting them to the wall of the tube using a magnet, removing the liquid by aspiration, replacing the liquid volume with the wash solution, removing the tube from the magnetic field, and agitating the tube to resuspend the microparticles.

Prior to metal-ion treatment, the magnetic microparticles were washed twice with 0.1 M MES buffer, pH 5.5 (containing 0.1% TRITON X-100) and then re-suspended in the same buffer. Following the wash step, 0.2 mL of 0.1 M gallium (III) nitrate, or ferric nitrate or zirconium (IV) nitrate in 0.01 M HCl solution per milligram of magnetic microparticles was added to the magnetic microparticle suspension. The mixture was allowed to shake gently for 1 h at room temperature and subsequently washed with the above MES buffer to remove excess metal ions. The resulting metal-ion mediated magnetic microparticles (Ga(III)-microparticles-1, Fe(III)-microparticles-1, Zr(IV)-microparticles-1, Ga(III)-microparticles-2, Fe(III)-microparticles-2, Zr(IV)-microparticles-2) were resuspended and stored at 4° C. in MES buffer. DYNABEADS MYONE Carboxylic Acid were used to prepare microparticles-1, and SERA-MAG Magnetic Particles were used to prepare microparticles-2.

### Example 2

### Metal Ion Comparison for DNA Capture and Release

In this experiment, 40 μg of Ga(III)-microparticles-1 and 40 μg of Fe(III)-microparticles-1) from Example 1 were used in separate experiments to bind 10⁵ cfu equivalent MRSA DNA (about 1.8 ng) in pH 5.5 MES buffer. The supernatant was designated SN0. The microparticles were then washed with MES buffer twice and each supernatant (designated SN1 and SN2, respectively) was collected. To elute the bound DNA, the microparticles were resuspended in 20 mM sodium phosphate buffer (PO₄, pH 8.5) and heated to 95° C. for 5 minutes. The supernatant (designated SN3) was collected for mecA-FAM RT-PCR analysis.

Five microliters of each sample (SN3) was subjected to real-time PCR amplification for mecA gene using the following optimized concentrations of primers, probe and enzyme, as well as thermo cycles. The sequence of all primers and probes listed below are given in the 5′→3′ orientation and are known and described in Francois, P., et al., Journal of Clinical Microbiology, 2003, volume 41, 254-260. The forward mecA primer was CATTGATCGCAACGTTCAATTT (SEQ ID NO:1). The mecA reverse primer was TGGTCTTTCTGCATTCCTGGA (SEQ ID NO:2). The mecA probe sequence, TGGAAGTTAGATTGGGATCATAGCGTCAT (SEQ ID NO:3), was dual labeled by 6-carboxyfluorescein (FAM) and IBFQ (IOWA BLACK FQ, Integrated DNA Technologies, Corniville, Iowa) at 5′- and 3′-position, respectively. PCR amplification was performed in a total volume of 10 mL containing 5 mL of sample and 5 mL of the following mixture: two primers (0.5 mL of 10 μM of each), probe (1 mL of 2 μM), MgCl₂ (2 mL of 25 mM) and LightCycler DNA Master Hybridization Probes (1 mL of 10×, Roche, Indianapolis, Ind.). Amplification was performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

The control samples consisted of DNA (equivalent to the amount used in the binding experiments) suspended in MES and phosphate buffers, respectively. The control DNA samples were not reacted with metal-ion mediated microparticles.

Table 1 shows the mecA PCR analysis data. The high cycle threshold (Ct) values (relative to control samples) in the SN0, SN1, and SN2 samples indicate the quantitative capture of the DNA. The similar Ct values (relative to control samples) in the SN3 samples indicate quantitative release of the captured DNA.

TABLE 1
PCR Analysis Data (The sample was suspended in 100 μL of
buffer and 5 μL of the resulting sample and 5 μL of PCR Master
mixture were used for PCR amplification.) Ct values are reported
from duplicate PCR reactions for each sample. A “Neg” result
indicates that there was no measurable Ct value in the 45 cycles
that were run.

| | |
|---|---|
| Sample | Cₜ |

| | | |
|---|---|---|
| Ga(III)MRSA + MES Wash-SN0 | 34.15 | 34.25 |
| Ga(III)MRSA + MES Wash-SN1 | Neg | Neg |
| Ga(III)MRSA + MES Wash-SN2 | 35.89 | 34.81 |
| Ga(III)MRSA + MES Wash-SN3 (PO₄) | 21.12 | 21.10 |
| Fe(III)MRSA + MES Wash-SN0 | 34.69 | 33.80 |
| Fe(III)MRSA + MES Wash-SN1 | 34.50 | Neg |
| Fe(III)MRSA + MES Wash-SN2 | 33.92 | 34.94 |
| Fe(III)MRSA + MES Wash-SN3 (PO₄) | 21.53 | 21.58 |
| 10⁵ MRSA Control-MES | 20.99 | 21.03 |
| 10⁵ MRSA Control-PO₄ | 20.39 | 20.49 |

### Example 3

### DNA Binding and Elution Efficiency Quantified by PicoGreen Assay

PicoGreen is a common method to quantify dsDNA in solution (Nakagawa, et al., Biotech & Bioeng. 2006, 94(5), 862-868). λDNA was chosen as a model to demonstrate the capture and release efficiency. λDNA, from the PicoGreen assay kit (Invitrogen, Carlsbad, Calif.), was diluted by 2-fold from 8 μg/mL to 0.25 μg/mL in 1×TE buffer (10 mM Tris-HCl, pH 8.0). 100 μL of each DNA solution was added to 100 μL of 0.1 M MES buffer (pH 5.5) containing 400 μg of Ga(III)-microparticles-2 and then well-mixed for 10 minutes. The microparticles were subsequently washed twice with MES buffer. 100 μl of 20 mM sodium phosphate buffer (pH 8.5) was added and the suspension was heated for 5 minutes at 65° C. to release the DNA from the microparticles.

In another experiment, the DNAs were first denatured at 95° C. for 5 minutes and put on ice immediately to generate single stranded DNA. The single stranded DNA was mixed with 400 μg of Ga(III)— microparticles-2 in MES at 0° C. for 10 minutes. After the microparticles were washed with MES twice, 100 μL of 20 mM PO₄ buffer was added to the microparticles and the suspension was heated at 65° C. for 5 minutes to release the DNA from the microparticles. The isolated phosphate supernatant (SN3) was again allowed to incubate at 65° C. for 1 h for DNA annealing. The re-formed dsDNA was quantified by the PicoGreen assay.

Table 2 shows the DNA binding and release data. 400 μg of Ga(III)-microparticles-2 can adsorb approximately 800 ng of ssDNA or dsDNA with about 94-99% capture efficiency. The second and fourth column (from left) in Table 2 demonstrates that both double stranded and single stranded DNA are eluted very efficiently from the microparticles.

TABLE 2
The capture/release efficiency of Ga(III)-microparticles-2 to
λDNA quantified by the PicoGreen assay. The results shown
below are the averages from triplicate assays.

| DNA | % Capture | % Recovery | % Capture | % Recovery |
|---|---|---|---|---|
| (μg) | (dsDNA) | (dsDNA) | (ssDNA) | (ssDNA) |
| 800 | 93.62 | 81.72 | 97.42 | 87.34 |
| 400 | 99.71 | 84.68 | 99.47 | 82.97 |
| 200 | 99.75 | 81.13 | 99.28 | 79.98 |
| 100 | 99.69 | 83.86 | 98.82 | 73.60 |
| 50 | 99.48 | 81.34 | 97.72 | 73.11 |
| 25 | 99.33 | 81.12 | 96.02 | 89.64 |

### Example 4

### Effect of Elution Buffers on DNA Release

DNA binding experiments were conducted in MES buffer or Tris (10 mM, pH 8.5) with Ga(III)-microparticles-1 described in Example 2. After the DNA binding process, the Ga(III)-microparticle/DNA complexes were washed twice with either MES buffer (0.1 M, pH 5.5), Tris buffer (10 mM, pH 8.5), or TAE buffer (40 mM Tris-acetate, 1 mM EDTA, pH 8.5) and eluted with Tris, TAE, or PO₄ buffer (20 mM sodium phosphate, pH 8.5). In some cases, the elution procedure included heating the suspension at 95° C. for 5 minutes. In other cases, the suspension was held at room temperature for 5 minutes to elute the DNA from the microparticles.

The supernatants (SN3) containing the eluted DNA were used for mecA RT-PCR analysis, as described in Example 2. Control samples were prepared as described in Example 2.

The resulting PCR analysis data shown in Table 3 indicate that both heating and the composition of the elution buffer can affect the efficiency of the DNA release from the beads. Heating increased the elution of DNA from the beads, except when water was used as the elution buffer. Both Tris and Tris-acetate buffers caused complete elution of the DNA in the presence of heat. A relatively smaller amount of DNA release, corresponding to higher Ct values, was observed at room temperature. Phosphate buffer provided for effective elution of DNA and was even more effective in combination with heat.

TABLE 3
The RT-PCR Ct values of the eluate from the mixture of 40 μg
of Ga(III)-microparticles-1 and 1.8 ng MRSA DNA (equivalent to
10⁵ cfu MRSA). Ct values are reported from duplicate
PCR reactions for each sample. Buffers used: Tris = 10 mM Tris-
HCl, pH 8.5; TAE = 40 mM Tris-acetate, 1 mM EDTA, pH 8.5;
PO₄ = 20 mM sodium phosphate, pH 8.5.

| Binding Buffer | Elution Buffer | Elution Temperature | Cₜ Value | |
|---|---|---|---|---|
| MES | Water | 95° C. | 28.08 | 28.19 |
| MES | Tris | 23° C. | 34.69 | 34.79 |
| MES | Tris | 95° C. | 20.66 | 20.47 |
| MES | TAE | 23° C. | 27.92 | 27.72 |
| MES | TAE | 95° C. | 19.80 | 19.74 |
| MES | PO₄ | 23° C. | 24.94 | 24.72 |
| Tris | Tris | 23° C. | 28.96 | 28.81 |
| Tris | Tris | 95° C. | 19.80 | 19.96 |
| Tris | PO₄ | 95° C. | 20.14 | 19.89 |
| — | Control | — | 20.13 | 20.15 |
| | (PO₄ buffer) | | | |
| — | Control | — | 19.76 | 20.06 |
| | (Tris buffer) | | | |

### Example 5

### Incubation Time for DNA Capture and Release

Incubation time for DNA capture and release may be an important parameter in certain processes such as microfluidic applications. 1.8 ng of DNA (equivalent to approximately 10⁵ cfu MRSA) was incubated with Ga(III)-microparticles-1, according to the procedure in Example 2, for various lengths of time ranging from 1 to 10 minutes. After the microparticles were washed by MES washing buffer, phosphate buffer (PO₄) was added to elute the bound DNA at 95° C. for various lengths of time ranging from 1 to 10 minutes. The supernatants were analyzed by mecA RT-PCR assay according to Example 2.

Table 4 shows the Ct values for the PCR assays. The results showed no difference in the Ct for samples that were allowed to bind for 1 from 10 minutes and were eluted for 10 minutes. Additionally, the data indicate that, for samples that were allowed to bind for 10 minutes, the DNA was quantitatively eluted within 1 minute in phosphate buffer at 95° C.

TABLE 4
Effect of binding and elution time on the recovery of DNA from
Ga(III)-microparticles. Ct values are reported for duplicate
experiments.

| | | |
|---|---|---|
| Binding Time | Elution Time | |
| (minutes) | (minutes) | Cₜ |

| | | | |
|---|---|---|---|
| 1 | 10 | 19.85 | 19.90 |
| 2 | 10 | 19.89 | 19.94 |
| 5 | 10 | 19.63 | 19.62 |
| 10 | 1 | 19.57 | 19.63 |
| 10 | 2 | 19.82 | 19.71 |
| 10 | 5 | 19.65 | 19.69 |
| 10 | 10 | 19.71 | 19.68 |

### Example 6

### MRSA DNA Dilution Series With Ga(III)-Microparticles-1 and Untreated Dynabeads Myone

Because the amount of DNA in a clinical sample load may be highly variable, capture and elution over a broad range of DNA concentrations may be useful. In this experiment, serial dilutions of MRSA DNA were bound to both Ga(III)-microparticles-1 and untreated DYNABEADS MYONE Carboxylic Acid magnetic beads (designated as “control”). Specifically, MRSA DNA was serially diluted by 10-fold from genome copies/mL (gc/mL) equivalents of 5×10⁶ cfu/mL to 5×10³ cfu/mL in 1×TEP buffer (10 mM Tris, 1 mM EDTA, pH 8.5, and 0.2% PLURONIC L64 (BASF, Mt. Olive, N.J.)). 10 μL of each MRSA DNA dilution was then added to 90 μL of 100 mM MES buffer (pH 5.5) containing 60 μg Ga(III)-microparticles. After gentle vortex for 15 minutes, the microparticle suspensions were washed and the supernatants were recovered as described in Example 2. After the second wash, the microparticles were resuspended in 100 μL 20 mM phosphate buffer (pH=8.5) and heated at 95° C. for 10 minutes. The heated microparticle mixture was immediately separated and final supernatant (SN3) was collected for RT-PCR analysis, using the mecA-FAM assay described in Example 2.

Table 5 shows the mecA-FAM PCR analysis data. All amounts of DNA eluted (SN3) from Ga(III)-microparticles showed similar Ct values to DNA control (in phosphate) samples, indicating the quantitative binding and release of the MRSA-specific DNA under these conditions. All of the SN0 (“unbound DNA”) supernatants showed primarily negative Ct values, indicating the ability of Ga(III)-microparticles to bind and elute over the range of DNA concentrations tested in these experiments. Additionally, all amounts of DNA eluted (SN3) from untreated microspheres showed primarily negative values (Ct values that were greater than or equal to 30), while the SN0 supernatants showed Ct values similar to the DNA control (phosphate) samples, indicating that very little DNA bound to carboxylated microparticles that were not pre-treated with the Ga(III) ions.

TABLE 5
Detection of MRSA DNA captured and eluted by Ga(III)-
microparticles and untreated carboxylated microparticles
using the mecA-FAM PCR assay. In some cases, Ct values
are reported for duplicate experiments.

| | | MRSA | | | |
|---|---|---|---|---|---|
| | | DNA | | | |
| | | (gene | cfu/reaction | | |
| Microparticles | Supernatant | copies) | (approx.) | Ct | |
| Ga³⁺- | SN0 | 5 × 10⁴ | 2500 | Neg | |
| Microparticles | | 5 × 10³ | 250 | Neg | |
| | | 5 × 10² | 25 | 35.37 | |
| | | 5 × 10¹ | 2.5 | Neg | |
| | SN3 | 5 × 10⁴ | 2500 | 22.68 | 22.88 |
| | | 5 × 10³ | 250 | 26.51 | 26.20 |
| | | 5 × 10² | 25 | 29.67 | 29.11 |
| | | 5 × 10¹ | 2.5 | 34.21 | 33.67 |
| No Ga³⁺ | SN0 | 5 × 10⁴ | 2500 | 22.72 | |
| Treatment | | 5 × 10³ | 250 | 26.35 | |
| | | 5 × 10² | 25 | 29.41 | |
| | | 5 × 10¹ | 2.5 | 32.53 | |
| | SN3 | 5 × 10⁴ | 2500 | 29.82 | 30.47 |
| | | 5 × 10³ | 250 | 33.54 | 33.37 |
| | | 5 × 10² | 25 | Neg | Neg |
| | | 5 × 10¹ | 2.5 | Neg | Neg |
| No | MES | 5 × 10⁴ | 2500 | 23.18 | |
| Microparticles | Buffer | 5 × 10³ | 250 | 26.97 | |
| (DNA | | 5 × 10² | 25 | 31.61 | |
| controls) | | 5 × 10¹ | 2.5 | 32.10 | |
| | PO₄ | 5 × 10⁴ | 2500 | 23.60 | |
| | Buffer | 5 × 10³ | 250 | 27.15 | |
| | | 5 × 10² | 25 | 30.57 | |
| | | 5 × 10¹ | 2.5 | 34.70 | |

### Example 7

### MSSA DNA Detection in the Presence of MRSE DNA

The ability to identify rare species from a complex sample, especially in the presence of another species with high DNA homology to the target species, may be useful. In this experiment, Methicillin-susceptible Staphylococcus aureus (MSSA) was analyzed in the presence of Methicillin-resistant Staphylococcus epidermidis MRSE. 10⁵ cfu equivalent of MSSA DNA was diluted by a factor of 10 in the presence of a constant amount of MRSE DNA (10⁵ cfu equivalent DNA). After incubating the DNA mixture with 40 μg Ga(III)-microparticles-1 in MES and washing twice with MES, bound DNA was released as in Example 6, and the phosphate buffer eluate was subject for RT-PCR assay. SAfemA PCR was performed to detect SAfemA gene present in MSSA. The procedure of running SAfemA PCR assay was carried out using the following optimized concentrations of primers, probe and enzyme, as well as thermo cycles. The sequence of all primers and probes listed below are given in the 5′→3′ orientation and are known. (See Francois, P., et al., Journal of Clinical Microbiology, 2003, volume 41, 254-260.) The forward SAfemA primer was TGCCTTACAGATAGCATGCCA (SEQ ID NO:4). The SAfemA reverse primer was AGTAAGTAAGCAAGCTGCAATGACC (SEQ ID NO:5). The SAfemA probe sequence, TCATTTCACGCAAACTGTTGGCCACTATG (SEQ ID NO:6), was dual labeled by fluorescein (FAM) and IBFQ at 5′- and 3′-position, respectively. PCR amplification was performed in a total volume of 10 μL containing 5 μL of sample and 5 μL of mixture of two primers (0.5 μL of 10 μM of each), probe (1 μL of 2 μM), MgCl₂ (2 μL of 25 mM) and LightCycler DNA Master Hybridization Probes (1 μL, 10×, Roche, Indianapolis, Ind.). Amplification was carried on LightCycler 2.0 (Roche) as follows: 95° C. for 30 s; 45 cycles of 95° C. for 0 s, 60° C. for 20 s. The mecA PCR assay, described in Example 2, was used to detect the mecA gene in MRSE.

Table 6 shows the Ct values for both assays. The data indicate that approximately 5 cfu MSSA can be detected in the presence of 5×10³ cfu of MRSE/reaction (5 μL of the 100 μL SN3 supernatant was used for the PCR reaction). The highest ratio of analyte/interfering species (i.e., MSSA:MRSE) detected in these experiments was approximately 1:1000. The Ct values for the DNA eluted from the microparticles consistently matched the Ct values from the control DNA mixtures (without microparticles). The presence of a consistent amount of MRSE in each sample was verified by the relatively constant Ct values from the mecA assays.

TABLE 6
The detection of MSSA genome in the presence of constant high
background (10⁵ genome copies) of MRSE DNA.

| | MSSA Assay | | MRSE Assay | |
|---|---|---|---|---|
| Sample | SAfemA Cₜ | SAfemA Cₜ | mecA Cₜ | mecA Cₜ |
| (gc MSSA) | (SN3) | (Control) | (SN3) | (Control) |
| 10⁵ | 22.08 | 22.03 | 22.08 | 21.80 |
| 10⁴ | 25.75 | 25.32 | 22.06 | 21.96 |
| 10³ | 29.00 | 28.88 | 22.15 | 22.12 |
| 10² | 32.39 | 32.68 | 22.05 | 21.84 |
| 10¹ | 35.75 | 36.16 | 22.24 | 21.89 |

### Example 8

### Detection of Internal Control Plasimid DNA

In genetic assays, an internal control (IC) test is commonly used to verify proper sample handling and functioning assay reagents, microfluidic transfer, and instrumentation. As the Ga(III)-microparticles are considered a reagent, it may be useful for the Ga(III)-microparticles to capture and release IC DNA, which is typically covalently closed, circular plasmid DNA. In this experiment, IC plasmid DNA, which was prepared by cloning SAfemA amplicons with a randomized SAfemA probe sequence used in SAfemA RT-PCR assay, was serially diluted by 10-fold from 10⁶ gc/mL to 10³ gc/mL in 1×TEP buffer. 10 μL, of each IC plasmid DNA dilution was added to 90 μL, of 100 mM MES buffer (pH 5.5) containing 60 μg Ga(III)-microparticles-1. After gentle vortex for 15 minutes, the microparticles were washed and the supernatants were collected as described in Example 2. After the second wash, the microparticles were resuspended in 100 μL 20 mM phosphate buffer, pH 8.5, and heated at 95° C. for 5 minutes. The heated microparticle mixture was immediately separated and SN3 supernatant was collected. All supernatants were assayed by using the same PCR protocol as described in Example 2. The same primers for SAfemA as described in Example 7 and a dual-labeled randomized probe sequence (TCATTTCACGCAAACTGTTGGCCACTATG) (SEQ ID NO:6) with FAM and IBFQ at 5′- and 3′-position, respectively for internal control DNA were used for the PCR amplification.

Table 7 shows the IC-SAfemA PCR analysis data. Samples eluted (SN3) from Ga(III)-microparticles showed similar Ct values to DNA control samples, indicating the capability of using Ga(III)-microparticles in these procedures to bind and elute SAfemA IC plasmid DNA.

TABLE 7
Detection of internal control (IC) plasmid DNA captured and eluted
by Ga(III)-microparticles using the IC-SAfemA PCR assay. In some
cases, Ct values are reported for duplicate experiments.

| | | IC-SAfemA | | | |
|---|---|---|---|---|---|
| | Supernatant | Plasmid DNA | (gc/ | | |
| Microparticles | (buffer) | (gc) | reaction) | Ct | |
| Ga³⁺- | SN3 | 10⁴ | 500 | 17.48 | 17.62 |
| microparticles | | 10³ | 50 | 22.28 | 22.19 |
| | | 10² | 5 | 25.36 | 25.25 |
| | | 10¹ | 0.5 | 29.68 | 29.73 |
| No | (PO₄ | 10⁴ | 500 | 18.68 | |
| Microparticles | Buffer) | 10³ | 50 | 23.72 | |
| (DNA control) | | 10² | 5 | 26.68 | |
| | | 10¹ | 0.5 | 29.13 | |

### Example 9

### MRSA Extraction and Subsequent Binding to Ga(III)-Microparticles

In this experiment, DNA was extracted from methicillin-resistant Staphylococcus aureus ATCC strain #BAA-43 (American Type Culture Collection; Manassas, Va.) (MRSA) using two extraction methods: a lysostaphin/proteinase K method or a lysostaphin-only method. The DNA released from these procedures was subsequently bound to and recovered from Ga(III)-microparticles-1. The control for this experiment consisted of DNA that was extracted from MRSA using the lysostaphin/proteinase K method without subsequent binding to Ga(III)-microparticles-1.

MRSA was grown overnight in Trypticase Soy Broth/0.2% PLURONIC L64 (TSBP) at 37° C. The overnight culture was then serially diluted by 10-fold from 2.3×10⁷ cfu/mL to 2.3×10³ cfu/mL in TEP buffer.

For the lysostaphin/proteinase K method, 66.7 μL of each MRSA dilution was treated with 26.7 μL of 250 μg/mL lysostaphin (Sigma Aldrich, St. Louis, Mo.) and held at room temperature for 5 minutes, after which 6.7 μL of 20 mg/mL proteinase K was added and the mixtures were incubated at 65° C. for 10 minutes and subsequently at 98° C. for 10 minutes. For the lysostaphin-only method, 66.7 μL of each MRSA dilution was mixed with 26.7 μL of 250 μg/mL lysostaphin and held at room temperature for 5 minutes. The DNA released from these procedures was then mixed with 6 μL of 100 mM MES buffer (pH 5.5) containing 60 μg Ga(III)-microparticles-1 (prepared as described in Example 1).

For the control method, 66.7 μL of each MRSA dilution was treated with the previously described lysostaphin/proteinase K method, without subsequent binding to Ga(III)-microparticles-1.

After gentle vortex for 5 minutes, the microparticle mixtures were separated and supernatants (SN0) were removed and discarded. The microparticles were then washed twice with 100 μL TEP buffer. After the second wash, the microparticles were resuspended in 100 μL 20 mM phosphate buffer (pH=8.5) and heated at 95° C. for 5 minutes, and the supernatants (SN3) were collected for RT-PCR analysis using the mecA-FAM assay as described above.

Table 8 shows the mecA-FAM PCR analysis data. The control DNA samples from the extraction method showed an irregular dose response Ct trend (the expected approximately 3.32 Ct shift for each 1:10 dilution was not observed). As compared to the control DNA samples, samples eluted (SN3) from microparticles that were reacted with DNA from the lysostaphin/proteinase K method showed an improved, more consistent dose response Ct trend (the expected approximately 3.32 Ct shift for each 1:10 dilution was observed). Whereas, samples eluted (SN3) from microparticles that were reacted with DNA from the lysostaphin-only method showed a shifted, irregular dose response Ct trend (the expected approximately 3.32 Ct shift for each 1:10 dilution was not observed, and the Ct values for each 1:10 dilution point are shifted from expected values).

TABLE 8
Detection of MRSA-extracted DNA captured and eluted by Ga(III)-
microparticles using the mecA-FAM PCR assay. Ct values are
reported for duplicate experiments.

| | Supernatant | MRSA | (cfu/ | mecA-FAM | |
|---|---|---|---|---|---|
| Treatment (buffer) | (buffer) | (cfu) | Reaction) | assay Ct | |
| Lysostaphin/ | n/a | 1,520,000 | 76,000 | 16.72 | 16.65 |
| Proteinase | (TEP) | 152,000 | 7,600 | 23.64 | 23.79 |
| K only (control) | | 15,200 | 760 | 28.54 | 28.26 |
| | | 1,520 | 76 | 30.92 | 30.82 |
| | | 152 | 8 | 33.28 | 33.17 |
| Lysostaphin/ | SN3 | 1,520,000 | 76,000 | 17.60 | 17.69 |
| Proteinase K, | (Phosphate) | 152,000 | 7,600 | 20.08 | 20.18 |
| then Ga(III) | | 15,200 | 760 | 23.54 | 23.55 |
| microparticles-1 | | 1,520 | 76 | 26.81 | 27.01 |
| | | 152 | 8 | 30.79 | 30.64 |
| Lysostaphin-only, | SN3 | 1,520,000 | 76,000 | 25.95 | 25.89 |
| then Ga(III) | (Phosphate) | 152,000 | 7,600 | 27.90 | 27.90 |
| microparticles-1 | | 15,200 | 760 | 29.76 | 29.68 |
| | | 1,520 | 76 | 34.00 | neg |
| | | 152 | 8 | neg | neg |

### Example 10

### Simultaneous MRSA Extraction and Binding to Ga(III)-Microparticles with Lysostaphin

Simultaneous extraction of the inputted sample and binding to Ga(III)-microparticles-1 in a single microfluidic chamber may be useful. In this experiment, DNA was simultaneously extracted from methicillin-resistant Staphylococcus aureus (MRSA) ATCC BAA-43 and bound to Ga(III)-microparticles-1, with and without a subsequent proteinase K treatment. These simultaneous extraction and binding methods were compared to a control method of lysostaphin/proteinase K extraction, followed by binding to Ga(III)-microparticles-1, as described in Example 9.

MRSA was grown overnight as described in Example 9. The overnight culture was then serially diluted by 10-fold from 1.4×10⁶ cfu/mL to 1.4×10² cfu/mL in TEP buffer.

For the control method, 66.7 μL, of each MRSA dilution was treated with the lysostaphin/proteinase K method, with subsequent binding to Ga(III)-microparticles-1, as described in Example 9. For the Sequential Lysis/DNA Binding/Digestion method, 66.7 μL of each MRSA dilution was mixed with 26.7 μL of 250 μg/mL lysostaphin, held at room temperature for 5 minutes, mixed with 6 μL of 100 mM MES buffer (pH 5.5) containing 60 μg Ga(III)-microparticles-1 (prepared as described in Example 1), gently vortexed at room temperature for 5 minutes, mixed with 6.7 μL proteinase K, incubated at 65° C. for 10 minutes and subsequently at 98° C. for 10 minutes. For the Simultaneous Lysis and DNA Binding method, 26.7 μL of 250 μg/mL lysostaphin was mixed with 6 μL of 100 mM MES buffer (pH 5.5) containing 60 μg Ga(III)-microparticles-1 and gently vortexed at room temperature for 5 minutes. This mixture was then added to 66.7 μL of each MRSA dilution and gently vortexed at room temperature for 5 minutes.

After gentle vortex for 5 minutes, the microparticle mixtures were washed twice, the DNA was eluted with phosphate buffer, and the final supernatants (SN3) were collected according to the methods in Example 9. All samples were then amplified and quantified by RT-PCR, using the mecA-FAM assay, as described in Example 2.

Table 9 shows the mecA-FAM PCR analysis data. Samples eluted (SN3) from Simultaneous Lysis and DNA Binding samples showed similar Ct results to Sequential Extraction/DNA Binding samples, indicating lysis of bacteria and binding to the microparticles can be completed in a single step. In addition, samples eluted (SN3) from Simultaneous Lysis and DNA Binding samples showed similar Ct results to Sequential Lysis/DNA Binding/Digestion samples, indicating proteinase K is not necessary for extraction and binding to Ga(III)-microparticles-1 with lysostaphin.

TABLE 9
Detection of MRSA-extracted DNA captured and eluted by
Ga(III)-microparticles-1 with lysostaphin using the mecA-FAM
PCR assay. Ct values are reported for duplicate experiments.

| | MRSA | | | |
|---|---|---|---|---|
| Treatment | (cfu) | (cfu/Reaction) | Ct | |
| Sequential | 92,000 | 4,600 | 20.48 | 20.64 |
| Extraction/DNA | 9,200 | 460 | 23.82 | 23.89 |
| Binding | 920 | 46 | 27.33 | 27.78 |
| | 92 | 5 | 31.59 | 31.17 |
| | 9 | 0.5 | neg | Neg |
| Simultaneous Lysis | 92,000 | 4,600 | 19.84 | 19.82 |
| and DNA Binding | 9,200 | 460 | 23.00 | 23.05 |
| | 920 | 46 | 26.65 | 26.65 |
| | 92 | 5 | 30.03 | 30.09 |
| | 9 | 0.5 | 32.80 | 33.86 |
| Sequential | 92,000 | 4,600 | 19.84 | 19.87 |
| Lysis/DNA | 9,200 | 460 | 23.00 | 23.00 |
| Binding/Digestion | 920 | 46 | 26.49 | 26.63 |
| | 92 | 5 | 30.19 | 29.93 |
| | 9 | 0.5 | 34.09 | 33.20 |

### Example 11

### MRSA Culture, Ga(III)-Microparticles vs. MagNA Pure

In this experiment, simultaneously lysing MRSA and binding MRSA DNA using Ga(III)-microparticles-2 is directly compared with the Roche MagNA Pure LC system using the MagNA Pure LC DNA Isolation Kit III (Bacteria, Fungi) kit (instrument and reagents obtained from Roche Diagnostics, Indianapolis, Ind.) for nucleic acid isolation from MRSA pure culture. MRSA (ATCC #BAA-43) was grown overnight as described in Example 9. The overnight culture was then serially diluted by 10-fold from 1.3×10⁷ cfu/mL to 1.3×10² cfu/mL in TEP buffer.

For MagNA Pure samples, the manufacturer's instructions for DNA purification were followed except that the following additional step was added to improve DNA recovery from the bacteria: 80 μL of each MRSA dilution was mixed with 20 μL of 250 μg/mL lysostaphin and incubated at 37° C. for 30 minutes. The 100 μL samples were then bound with 130 μL bacterial lysis buffer and 20 μL of proteinase K (kit supplied) to 250 μL total input volume and eluted to 100 μL elution volume after the completion of DNA extraction, according to the manufacturer's instructions.

For Simultaneous Lysis and DNA Binding samples, 80 μL of each MRSA dilution was mixed with 10 μL of 100 mM MES buffer (pH 5.5) containing 100 μg Ga(III)-microparticles-2 pre-mixed with 26.7 μL of 250 μg/mL lysostaphin, as in Example 10. After gentle vortex for 5 minutes, the microparticle mixtures were washed twice, the DNA was eluted with phosphate buffer, and the final supernatants (SN3) were collected according to the methods in Example 9. All samples were then amplified and quantified by RT-PCR, using the mecA-FAM assay, as described in Example 2.

Table 10 shows the mecA-FAM PCR analysis data. Samples eluted (SN3) from Simultaneous Lysis and DNA Binding samples showed consistently lower Ct results than MagNA Pure samples, indicating the Simultaneous Lysis and Binding method captured and/or released the DNA more efficiently than the adapted-MagNA Pure method.

TABLE 10
Comparison of simultaneous lysis and DNA binding to Ga(III)-
microparticles-2 vs. MagNA Pure as methods for nucleic acid
isolation from MRSA pure culture using the mecA-FAM PCR
assay. Ct values are reported for duplicate experiments.

| Sample | Supernatant | MRSA | (cfu/ | | |
|---|---|---|---|---|---|
| Treatment | (buffer) | (cfu) | Reaction) | Ct | |
| (Lysostaphin + | SN3 | 1,060,000 | 53000 | 16.44 | 16.31 |
| Ga³⁺ | (Phosphate) | 106,000 | 5300 | 19.86 | 19.76 |
| Microparticles- | | 10,600 | 530 | 23.00 | 23.85 |
| 2) | | 1,060 | 53 | 26.53 | 26.98 |
| | | 106 | 5.3 | 30.71 | 30.48 |
| | | 11 | 0.5 | 33.06 | 35.61 |
| MagNA Pure | n/a | 1,060,000 | 53000 | 17.89 | 18.07 |
| | (Roche Kit | 106,000 | 5300 | 21.16 | 21.67 |
| | Elution | 10,600 | 530 | 24.85 | 25.24 |
| | Buffer) | 1,060 | 53 | 28.64 | 28.54 |
| | | 106 | 5.3 | 32.14 | 31.94 |
| | | 11 | 0.5 | 33.82 | 32.94 |
| No Template | TEP | n/a | N/a | neg | neg |
| Control (NTC) | Phosphate | | | neg | neg |

### Example 12

### Extraction and Detection of aureus (SA) from Clinical Nasal Swab Samples Using Ga(III)-Microparticles vs. MagNA Pure

For clinical swab samples, overcoming PCR inhibitors, for example, in nasal mucous during capture and elution can be useful. In this experiment, the Simultaneous Lysis and DNA Binding procedures of Example 10 were used to capture and elute known SA-positive swab samples from two different patients, verified by a microbiology culture method.

Two patients were chosen for S. aureus studies. The specimen was collected from a nostril with a general swab and kept at −80° C. prior to studies (two swabs for each patient referred to as 1-1, 1-5 and 2-1, 2-5). Culture studies showed that these two patients were S. aureus positive. Each nasal swab sample was first eluted by 410 μL TEP solution by vortexing for 60 seconds. For each test, 80 μL of the swab eluate was combined with 160 μL of liquid containing 100 μg of Ga(III)-microparticles-2 and 9 μg of lysostaphin in TEP. The mixture was incubated at room temperature for 5 minutes with occasional gentle shaking and then magnetically separated. The supernate was discarded and the remaining microparticles were washed twice by 100 μL TEP. Finally, the microparticles were resuspended in 100 μL of 20 mM phosphate buffer (pH 8.5) and heated at 97° C. for 10 minutes. The resulting supernate was magnetically separated and used for PCR analysis.

For control MagNA pure samples, culture MRSA sample was diluted by a factor of 10 from 148,000 cfu to 148 cfu in 80 μL TEP. To each MRSA sample, 5 ng of lysostaphin was added and incubated at 37° C. for 30 min after gentle mixing. 130 μL of Bacteria Lysis Buffer (MagNA Pure LC DNA Isolation Kit III) and 20 μL of Proteinase K (supplied with same kit) were then added to the sample with gentle mixing, followed by incubating at 95° C. for 10 minutes. DNA extraction was completed by following by the manufacturer's instruction on Roche's MagNA Pure LC instrument.

SA-femA qPCR analysis was completed as in Example 7.

In Table 11, the data acquired from this experiment showed that the Ct values obtained from both Ga (III)-microparticles-2 and MagNA pure methods were very close. No significant inhibitory effects were observed from these two patient samples. According to the reference numbers of MRSA, each swab bears roughly around 1.4×10⁵ cfu of S. aureus.

TABLE 11
Detection of spiked MRSA-extracted DNA captured and eluted
by Ga(III)-microparticles-2 with lysostaphin from nasal swab samples
(known SA positive from culture) using the SAfemA-FAM PCR assay.
Ct values are reported for duplicate experiments.

| Sample | Swab description | | | Ct values of SAfemA- | |
|---|---|---|---|---|---|
| Type | (patient-swab #) | Treatment | cfu/rxn | FAM-qPCR assay | |
| Patient | 1-1 | Simultaneous Method | unknown | 24.04 | 24.04 |
| Nasal | | MagNA Pure | unknown | 24.72 | 25.32 |
| Swab | 1-5 | Simultaneous Method | unknown | 25.74 | 25.90 |
| | | MagNA Pure | unknown | 25.06 | 26.80 |
| | 2-1 | Simultaneous Method | unknown | 22.59 | 22.54 |
| | | MagNA Pure | unknown | 23.63 | 23.71 |
| | 2-5 | Simultaneous Method | unknown | 23.92 | 23.84 |
| | | MagNA Pure | unknown | 25.91 | 25.49 |
| MRSA | 148,000 cfu | MagNA Pure | 7,400 | 22.81 | 22.99 |
| Culture | 14,800 cfu | | 740 | 26.14 | 26.09 |
| | 1,480 cfu | | 74 | 29.64 | 29.82 |
| | 148 cfu | | 7 | 33.17 | 34.16 |

### Example 13

### MRSA Binding Onto Ga(III)-Microparticles And Zr(IV)-Microparticles

In this experiment, MRSA was captured onto Ga(III)-microparticles-2 or Zr(IV)-microparticles-2 in TEP or 100 mM MES (pH 5.5)/0.2% PLURONIC L64 (MESP) buffers using a 1 mL reaction volume. Ga(III)-microparticles-2 or Zr(IV)-microparticles-2 were prepared as in Example 1.

MRSA was grown overnight in TSBP broth as described in Example 9. The overnight culture was then serially diluted by 10-fold to final concentrations of approximately 1.5×10³ cfu/mL and 1.5×10² cfu/mL, respectively, in TEP buffer. For TEP and MESP samples, 10 μL of each MRSA dilution was further diluted with 990 μL TEP or MESP buffer, respectively. For MRSA capture, 10 μL MES buffer containing 100 μg Ga(III)-microparticles-2 or Zr(IV)-microparticles-2 was added to each sample, respectively, and the mixture was gently vortexed for 15 minutes at room temperature. The microparticle mixtures were separated, washed twice, resuspended, and the MRSA in each suspension was quantified by plating appropriate volumes of each solution onto blood agar plates, incubating the plates at 37° C. for 18 hours, and subsequent enumeration of the colonies.

Table 12 shows the resulting plate count data. Bacteria capture onto both Ga(III)-microparticles-2 and Zr(IV)-microparticles-2 was improved at low pH (MES) buffer conditions. Specifically, Ga(III)-microparticles-2 show negligible bacteria capture in TEP buffer, but show 99% bacteria capture in MES buffer. And Zr(IV)-microparticles-2 show 89% bacteria capture in TEP buffer, but show 100% bacteria capture in MES buffer.

TABLE 12
Plate count data for MRSA binding onto Ga(III)-microparticles-2
and Zr(IV)-microparticles-2 in TEP or MESP buffers using a
1 mL reaction volume. The SPIKE solution shows the number of
bacteria in the original washed bacterial suspension.

| | | | Total | |
|---|---|---|---|---|
| Buffer | Beads | Plating sample | cfu | % Capture |
| TEP | No beads control | Spike bacteria | 1150 | n/a |
| | Ga³⁺-microspheres-2 | SN0 | 895 | 89.8 |
| | | SN1 | 70 | 7.0 |
| | | SN2 | 22 | 2.2 |
| | | Bacteria + Beads | 10 | 1.0 |
| | Zr⁴⁺-microspheres-2 | SN0 | 215 | 10.1 |
| | | SN1 | 14 | 0.7 |
| | | SN2 | 0 | 0.0 |
| | | Bacteria + Beads | 1890 | 89.2 |
| MESP | No beads control | Spike bacteria | 1790 | n/a |
| | Ga³⁺-microspheres-2 | SN0 | 25 | 1.4 |
| | | SN1 | 0 | 0.0 |
| | | SN2 | 0 | 0.0 |
| | | Bacteria + Beads | 1770 | 98.6 |
| | Zr⁴⁺-microspheres-2 | SN0 | 0 | 0 |
| | | SN1 | 0 | 0 |
| | | SN2 | 0 | 0 |
| | | Bacteria + Beads | 2320 | 100 |

### Example 14

### MRSA Binding, Lysis, and DNA Capture onto Ga(III)-Microparticles and Zr(IV)-Microparticles

In this experiment, MRSA was captured onto Ga(III)-microparticles-2 or Zr(IV)-microparticles-2, lysed (on the microparticles) with an enzyme to release the bacterial DNA, and the DNA was recaptured onto the same microparticles. Subsequently, the DNA was eluted from the microparticles for quantitation by mecA-FAM RT-PCR procedure described in Example 2.

MRSA was grown overnight and serially diluted by 10-fold from 2.0×10⁷ cfu/mL to 2.0×10³ cfu/mL in TEP buffer, as in Example 11. Aliquots (10 μL) of each MRSA dilution were further diluted with 990 μL MESP buffer and were mixed with 10 μL of MES buffer containing 100 μg Ga(III)-microparticles-2 or Zr(IV)-microparticles-2 microparticles and gently vortexed at room temperature for 5 minutes and washed as described in Example 13. Next, 26.7 μL of 250 μg/mL lysostaphin was added and the mixture was gently vortexed at room temperature for 5 minutes. This method is referred as Sequential Method.

For control samples, MRSA was simultaneously lysed and the released DNA bound onto Ga(III)-microparticles-2 (Simultaneous Method). Lysostaphin, 26.7 μL of 250 μg/mL, was mixed with 10 μL of MES buffer containing 100 μg Ga(III)-microparticles-2 microparticles and gently vortexed at room temperature for 5 minutes. This mixture was then added to 10 μL of each MRSA dilution further diluted with 90 μL TEP buffer and gently vortexed at room temperature for 5 minutes.

After gentle vortex for 5 minutes, the microparticle mixtures for both methods were separated and supernatants (SN0) were removed and discarded. The microparticles were then washed twice with 100 μL TEP buffer, as described in Example 13. After the second wash, the microparticles were resuspended in 100 μL phosphate buffer, heated at 95° C. for 10 minutes, and separated, and then the supernatants (SN3) were collected for mecA-FAM RT-PCR analysis, as described in Example 2.

Table 13 shows the mecA-FAM RT-PCR quantitative analysis data. Eluate from Sequential Method samples showed similar Ct results to Simultaneous Method samples, indicating bacteria were sequentially captured onto and then lysed on the microparticles, and then the released DNA was recaptured onto the same microparticles. In addition, eluate from Sequential Method samples with Zr(IV)-microparticles-2 consistently showed slightly lower Ct results than Sequential Method samples with Ga(III)-microparticles-2, indicating Zr(IV)-microparticles may more effectively capture bacteria and/or DNA.

TABLE 13
Detection of DNA eluate from Ga(III)-microparticles-2 or Zr(IV)-
microparticles-2 after MRSA was sequentially captured onto
and lysed on the microparticles, and then the released DNA was
recaptured onto the same microparticles using mecA-FAM RT-PCR.

| | | | | | |
|---|---|---|---|---|---|
| | MRSA | Bacteria Capture | DNA capture | | mecA-FAM assay |
| Method | (cfu) | Beads | beads | (cfu/rxn) | Ct |

| | | | | | |
|---|---|---|---|---|---|
| Sequential | 202,000 | Ga(III)-microparticles-2 | 10,100 | 21.14 | 21.15 |
| | | Zr(IV)-microparticles-2 | | 19.94 | 19.90 |

| | | | | |
|---|---|---|---|---|
| Simultaneous | n/a | Ga(III)- | 20.86 | 20.84 |
| | | microparticles-2 | | |

| | | | | | |
|---|---|---|---|---|---|
| Sequential | 20,200 | Ga(III)-microparticles-2 | 1,010 | 24.91 | 25.01 |
| | | Zr(IV)-microparticles-2s | | 23.29 | 23.32 |

| | | | | |
|---|---|---|---|---|
| Simultaneous | n/a | Ga(III)- | 24.68 | 24.62 |
| | | microparticles-2 | | |

| | | | | | |
|---|---|---|---|---|---|
| Sequential | 2,020 | Ga(III)-microparticles-2 | 101 | 28.24 | 27.80 |
| | | Zr(IV)-microparticles-2 | | 26.77 | 26.70 |

| | | | | |
|---|---|---|---|---|
| Simultaneous | n/a | Ga(III)- | 28.17 | 28.25 |
| | | microparticles-2 | | |

| | | | | | |
|---|---|---|---|---|---|
| Sequential | 202 | Ga(III)-microparticles-2 | 10 | 31.47 | 31.79 |
| | | Zr(IV)-microparticles-2 | | 29.74 | 30.56 |

| | | | | |
|---|---|---|---|---|
| Simultaneous | n/a | Ga(III)- | 30.65 | 30.92 |
| | | microparticles-2 | | |

| | | | | | |
|---|---|---|---|---|---|
| Sequential | 20 | Ga(III)-microparticles-2 | 1 | 34.09 | 36.08 |
| | | Zr(IV)-microparticles-2 | | 33.72 | 34.77 |

| | | | | |
|---|---|---|---|---|
| Simultaneous | n/a | Ga(III)- | 34.34 | 36.04 |
| | | microparticles-2 | | |

### Example 15

### MRSA Binding onto Ga(III)-Microparticles

In this experiment, MRSA (ATCC BAA-43) was captured onto Ga(III)-microparticles in TEP. Ga(III)— microparticles-2 were prepared as in Example 1.

MRSA was grown overnight in TSBP broth as described in Example 9. The overnight culture was then serially diluted by 10-fold to final concentrations of approximately 1.5×10³ cfu/mL and 1.5×10² cfu/mL, respectively, in TEP buffer. For MRSA capture, 10 μL MES buffer containing 100 μg Ga(III)-microparticles-2 was added to 10 mL of each MRSA dilution, respectively, and the mixtures were gently vortexed for 15 minutes at room temperature. The microparticle mixtures were separated, and the supernatants were removed (SN0). The microparticles were washed twice with 100 μL TEP buffer, vortexing, separating, and removing the supernatants (SN1 and SN2). After the second wash, the microparticles were resuspended in 100 μL of 20 mM Phosphate Buffer ((pH of 8.5) (PB buffer). The captured MRSA and the MRSA in each supernatant were quantified by plating appropriate volumes of each solution onto blood agar plates, incubating the plates at 37° C. for 18 hours, and subsequent enumeration of the colonies.

Table 14 shows the resulting plate count data. Ga(III)-microparticles-2 captured approximately 26% bacteria at 1.5×10³ cfu and 30% bacteria at 1.5×10² cfu.

TABLE 14
Plate count data for MRSA binding onto Ga(III)-microparticles-2
in TEP buffer using a 1 mL reaction volume. The SPIKE solution
shows the number of bacteria in the original washed
bacterial suspension.

| | | | | Ave. Plate | | |
|---|---|---|---|---|---|---|
| | Plating | | | Count | Calculated | |
| cfu | sample | Sample (μL) | Plate (μL) | (cfu) | Total cfu | % Capture |
| 1.5 × 10³ | Spike bacteria | n/a | 100 | 147 | 1470 | n/a |
| | SN0 | 1000 | 200 | 139 | 695 | 60.1 |
| | SN1 | 100 | 100 | 126 | 126 | 10.9 |
| | SN2 | 100 | 100 | 36 | 36 | 3.1 |
| | Bacteria + | 1000 | 100 | 30 | 300 | 25.9 |
| | microparticles | | | | | |
| 1.5 × 10² | Spike bacteria | n/a | 100 | 147 | 147 | n/a |
| | SN0 | 1000 | 200 | 16 | 80 | 62.5 |
| | SN1 | 100 | 100 | 8 | 8 | 6.3 |
| | SN2 | 100 | 100 | 2 | 2 | 1.6 |
| | Bacteria + | 100 | 100 | 38 | 38 | 29.7 |
| | microparticles | | | | | |

### Example 16

### MRSA Binding onto Ga(III)-Microparticles and Zr(IV)-Microparticles

In this experiment, MRSA was captured onto Ga(III)-microparticles-2 or Zr(IV)-microparticles-2 in TEP and 10 mM Tris-HCl (pH 8.5)/0.2% PLURONIC L64 (TP) buffers using a 1 mL reaction volume. Ga(III)-microparticles-2 or Zr(IV)-microparticles-2 were prepared as in Example 1.

MRSA was grown overnight in TSBP broth as described in Example 9. The overnight culture was then serially diluted by 10-fold to final concentrations of 1.5×10³ cfu/mL in TEP buffer and 2.3×10³ cfu/mL in TP buffer. For MRSA capture, 10 μL MES buffer containing 100 μg Ga(III)-microparticles-2 or Zr(IV)-microparticles-2 was added to 1 mL of each MRSA dilution, respectively, and the mixture was gently vortexed for 15 minutes at room temperature. The microparticle mixtures were separated, and the supernatants were removed (SN0). The microparticles were washed twice with 100 μL TEP or TP buffer, respectively, vortexing, separating, and removing the supernatants (SN1 and SN2). After the second wash, the microparticles were resuspended in 100 μL of 20 mM Phosphate Buffer ((pH of 8.5) (PB buffer). The captured MRSA and the MRSA in each supernatant were quantified by plating appropriate volumes of each solution onto blood agar plates, incubating the plates at 37° C. for 18 hours, and subsequent enumeration of the colonies.

Table 15 shows the resulting plate count data. Both Ga(III)-microparticles-2 and Zr(IV)-microparticles-2 captured bacteria more efficiently in TEP buffer.

TABLE 15
Plate count data for MRSA binding onto Ga(III)-microparticles-2 and Zr(IV)-
microparticles-2 in TEP or TP buffers using a 1 mL reaction volume. The
SPIKE solution shows the number of bacteria in the original washed
bacterial suspension. The projected count was 10³ cfu.

| | Ga(III) | | | | Ave Plate | | |
|---|---|---|---|---|---|---|---|
| | or | Plating | Sample | | Count | Calculated | |
| Buffer | Zr(IV) | sample | (μL) | Plate (μL) | (cfu) | Total cfu | % Capture |
| TEP | n/a | Spike | n/a | 100 | 146 | 1460 | n/a |
| | | bacteria | | | | | |
| | Ga(III) | SN0 | 1000 | 200 | 188 | 940 | 73.9 |
| | | SN1 | 100 | 100 | 62 | 62 | 14.9 |
| | | SN2 | 100 | 100 | 40 | 40 | 3.1 |
| | | Bacteria + | 1000 | 100 | 23 | 230 | 18.1 |
| | | Beads | | | | | |
| | Zr(IV) | SN0 | 1000 | 200 | 98 | 490 | 22.0 |
| | | SN1 | 100 | 100 | 0 | 0 | 0.0 |
| | | SN2 | 100 | 100 | 0 | 0 | 0.0 |
| | | Bacteria + | 1000 | 100 | 174 | 1740 | 78.0 |
| | | Beads | | | | | |
| TP | n/a | Spike | n/a | 100 | 228 | 2280 | n/a |
| | | bacteria | | | | | |
| | Ga(III) | SN0 | 1000 | 200 | 278 | 1390 | 76.5 |
| | | SN1 | 100 | 100 | 147 | 147 | 8.1 |
| | | SN2 | 100 | 100 | 51 | 51 | 2.8 |
| | | Bacteria + | 1000 | 100 | 23 | 230 | 12.7 |
| | | Beads | | | | | |
| | Zr(IV) | SN0 | 1000 | 200 | 197 | 985 | 53.8 |
| | | SN1 | 100 | 100 | 29 | 29 | 1.6 |
| | | SN2 | 100 | 100 | 8 | 8 | 0.4 |
| | | Bacteria + | 1000 | 100 | 81 | 810 | 44.2 |
| | | Beads | | | | | |

### Example 17

### MRSA Binding onto and Release from Ga(III)-Microparticles

In this experiment, MRSA (ATCC BAA-43) was captured onto Ga(III)-microparticles-2 in MESP buffer using a 1 mL reaction volume and then subsequently released from the beads using a high pH and/or competing reagent buffer. Ga(III)-microparticles-2 were prepared as in Example 1.

MRSA was grown overnight in TSBP broth as described in Example 9. The overnight culture was then serially diluted by 10-fold to final concentrations of approximately 2.04×10⁴ cfu/mL in TEP buffer. For MRSA capture, 10 μL of MRSA dilution was mixed with 990 μL 100 mM MES (pH 5.5)/0.2% PLURONIC L64 (MESP) buffer) and 10 μL MES buffer containing 100 μg Ga(III)-microparticles, and the mixtures was gently vortexed for 15 minutes at room temperature. The microparticle mixtures were separated, and the supernatants was removed. The microparticles were washed twice with 100 μL MESP buffer, vortexing, separating, and removing the supernatants. After the second wash, the microparticles were resuspended in 100 μL of 100 mM Phosphate Buffer (pH 7.0)/0.2% PLURONIC L64, 100 μL of 100 mM Phosphate Buffer (pH 9.5)/0.2% PLURONIC L64, 100 μL of 10 mM Tris-HCl(pH of 9.5)/0.2% PLURONIC L64, or 100 μL of 10 mM EDTA (pH 8.0)/0.2% PLURONIC L64 by vortexing. To estimate the captured MRSA on microparticles, appropriate volumes of the microparticle mixtures were plated onto blood agar plates. To estimate released MRSA from the microparticles, the microparticle mixture was separated and the supernatants (SN3) were quantified by plating appropriate volumes of each supernatant onto blood agar plates, incubating the plates at 37° C. for 18 hours, and subsequent enumeration of the colonies.

Table 16 shows the resulting plate count data. The 10 mM EDTA (pH 8.0)/0.2% PLURONIC L64 showed the best MRSA release from the Ga(III)-microparticles-2, which released 24.6% MRSA from the microparticles into the supernatant (SN3).

TABLE 16
Plate count data for MRSA release from Ga(III)-microparticles-2
in 100 mM Phosphate Buffer (pH 7.0)/0.2% PLURONIC L64,
100 mM Phosphate Buffer (pH 9.5)/0.2% PLURONIC L64, 10 mM
Tris-HCl(pH of 9.5)/0.2% PLURONIC L64, or 10 mM
EDTA (pH 8.0)/0.2% PLURONIC L64

| | | | | Ave. Plate | | |
|---|---|---|---|---|---|---|
| Elution | | | Volume | Count | Calculated | % |
| Buffer | pH | Sample | (μL) | (cfu) | Total cfu | Release |
| TEP | 8.0 | 10⁶ cfu | n/a | 204 | 204 | n/a |
| Phosphate | 9.5 | MRSA | n/a | 187 | 187 | n/a |
| Phosphate | 7.0 | | n/a | 215 | 215 | n/a |
| Tris-HCl | 9.5 | | n/a | 237 | 237 | n/a |
| EDTA | 8.0 | | n/a | 178 | 178 | n/a |
| Phosphate | 9.5 | SN3 | 1000 | 4 | 4 | 2.0 |
| Phosphate | 7.0 | | 800 | 3 | 3 | 1.2 |
| Tris-HCl | 9.5 | | 800 | 7 | 7 | 3.4 |
| EDTA | 8.0 | | 800 | 50 | 50 | 24.6 |
| Phosphate | 9.5 | Ga(III) + | 700 | 246 | 172 | n/a |
| Phosphate | 7.0 | SN3 | 1000 | 214 | 214 | n/a |
| Tris-HCl | 9.5 | | 1000 | 194 | 194 | n/a |
| EDTA | 8.0 | | 1000 | 201 | 201 | n/a |

### Example 18

### Capture of Yeast Cells by Fe(III)-Microparticles and Zr(IV)-Microparticles

An isolated colony of Candida albicans (ATCC MYA-2876) was inoculated into 10 ml Difco Sabouraud Dextrose broth (Becton Dickinson, Sparks, Md.) and incubated at 37° C. for 18-20 hours. This overnight culture at ±5×10⁷ cfu/mL was diluted in sterile Butterfield's Buffer solution (pH 7.2±0.2; monobasic potassium phosphate buffer solution; VWR Catalog Number 83008-093, VWR, West Chester, Pa.) to obtain a 100 cfu/mL dilution. Colony forming units (cfu) are units of live/viable yeast.

Apple juice (pasteurized) was purchased from local grocery store (Cub Foods, St. Paul). A volume of 11 ml apple juice was added to a sterile 250 mL glass bottle (VWR, West Chester, Pa.). A volume of 99 mL of sterile Butterfield's Buffer solution was added the apple juice. The contents were mixed by swirling for 1 minute. The diluted apple juice sample was spiked with Candida to obtain a final concentration of 50 cfu/ml using the above overnight culture. Spiked apple juice samples (1.0 mL) were added to labeled, sterile 5 mL polypropylene tubes (Falcon, Becton Dickinson, N.J.) containing 100 microgram of Ga(III)-microparticles-2, Fe(III)-microparticles-2, Zr(IV)-microparticles-2, and control SERA-MAG Magnetic Particles particles without metal ions, respectively, and mixed on a THERMOLYNE MAXIMIX PLUS vortex mixer (Barnstead International, Iowa) for 30 seconds. The capped tubes were incubated at room temperature (25° C.) for 20 minutes on a THERMOLYNE VARI MIX shaker platform (Barnstead International, Iowa). After the incubation, the beads were separated from the sample for 10 minutes by using a magnetic holder (Dynal, Carlsbad, Calif.). Control tubes containing 1.0 mL of 50 cfu/ml Candida, without any magnetic beads, were treated similarly. The supernatant (1 mL) was removed and plated onto PETRIFILM Yeast and Mold Count plates (dry, rehydratable culture medium from 3M Company, St. Paul., MN) and incubated for 5 days as per the manufacturers instructions. The separated magnetic beads were removed from the magnetic stand, resuspended in 1 mL sterile Butterfield's Buffer and plated on PETRIFILM Yeast and Mold Count plate (dry, rehydratable culture medium from 3M Company, St. Paul., MN) and incubated for 5 days as per the manufacturers instructions. Isolated yeast colonies were counted manually and % capture was calculated as number of colonies from plated magnetic beads divided by number of colonies in the plated untreated control multiplied by 100.

### CFU=Colony Forming Units is a Unit of Live/Viable Yeast

The Fe(III)-microparticles-2 and Zr(IV)-microparticles-2 bound and concentrated 67% and 81% (standard deviation <10%), respectively, the C. albicans cells from the sample. The control particles bound and concentrated 33% (standard deviation <10%) C. albicans cells from apple juice sample.

### Example 19

### Capture of Mold Cells by Ga(III)-Microparticles, Fe(III)-Microparticles, Zr(IV)-microparticles

Ga(III)-microparticles-2, Fe(III)-microparticles-2, Zr(IV)-microparticles-2, and corresponding microparticles without metal ions (25 μg each) were tested separately as described in Example 18, but for capture of spores of Aspergillus niger (ATCC 16404). Spore stock at concentration of about 1×10⁸ spores/mL was obtained from ATCC (The American Type Culture Collection (ATCC; Manassas, Va.). The results are shown in Table 17 below.

TABLE 17
Capture of Aspergilus niger by Ga(III)-microparticles-2, Fe(III)-
microparticles-2, Zr(IV)-microparticles-2, and corresponding
microparticles without metal ions.

| Microparticles | % Capture |
|---|---|
| Without metal ions | 88 |
| Fe(III)-microparticles-2 | 93 |
| Ga(III)-microparticles-2 | 98 |
| Zr(IV)-microparticles-2 | 100 |

Data are representative of two independent experiments.

### Example 20

### Capture of Salmonella by Ga(III)-Microparticles, Fe(III)-Microparticles and Zr(IV)-Microparticles from Food Samples

Food samples were purchased from a local grocery store (Cub Foods, St. Paul). Food samples (sliced ham/pureed bananas/apple juice) (11 g) were weighed in sterile dishes and added to sterile STOMACHER polyethylene filter bags (Seward Corp, Norfolk, UK). This was followed by the addition of 99 mL of Butterfield's Buffer solution to each food sample. The resulting samples were blended for a 2-minute cycle in a STOMACHER 400 Circulator laboratory blender (Seward Corp). The blended samples were collected in sterile 50 mL centrifuge tubes (BD FALCON, Becton Dickinson, Franklin Lakes, N.J.) and centrifuged at 2000 revolutions per minute (rpm) for 5 minutes to remove large debris. The resulting supernatants were used as samples for further testing.

Bacterial dilutions were prepared in solution (pH 7.2±0.2; monobasic potassium phosphate buffer solution (VWR Catalog Number 83008-093, VWR, West Chester, Pa.). The blended food samples were spiked with bacterial cultures at a 1.6−2.6×10² CFU/mL concentration using dilutions from an 18-20 hour overnight culture (˜1×10⁹ CFU/mL) of Salmonella enterica subsp.enterica serovar Typhimurium (ATCC 35987). Ga(III)-microparticles-2, Fe(III)-microparticles-2, and Zr(IV)-microparticles-2 were added to separate sterile 5 ml polypropylene tubes (Falcon, Becton Dickinson, N.J.) containing 1 ml of spiked supernatant. The metal ion coated magnetic particles were tested at a concentration of 100 μg/ml. The tubes were capped, contents were mixed on a THERMOLYNE MAXIMIX PLUS vortex mixer (Barnstead International, Iowa) and incubated at room temperature (25° C.) for 15 minutes. The capped tubes were incubated at room temperature (25° C.) for 20 minutes on a THERMOLYNE VARI MIX shaker platform (Barnstead International, Iowa). After the incubation, the magnetic particles were separated for 10 minutes using a magnet (Dynal, Carlsbad, Calif.). Control tubes containing 100 μg/ml of unmodified magnetic particles (1 micron diameter Seradyn carboxylic acid from Indianapolis, Ind.) without metal-ions were treated similarly. The supernatant (1 ml) was removed and plated onto PETRIFILM Aerobic Count Plates (3M Company, St. Paul., MN) as per the manufacturers instructions. The separated magnetic particles were resuspended in 1 ml Butterfield's Buffer and were plated on PETRIFILM Aerobic Count Plates. After 48 hrs incubation at 37° C., bacterial colonies were quantified using a PETRIFILM Plate Reader (3M Company, St. Paul., MN). The % capture was calculated as (Number of colonies from plated particles/Number of colonies in the plated untreated control)×100. The results are shown in Table 18 below.

TABLE 18
Capture of Salmonella by magnetic particles without and with
bound Ga(III), Fe(III), or Zr(IV) from food samples.

| Food Sample | Microparticles | % Capture |
|---|---|---|
| Apple Juice | Ga(III)-microparticles-2 | 48 |
| | Fe(III)-microparticles-2 | 74 |
| | Zr(IV)-microparticles-2 | 81 |
| Ham | Ga(III)-microparticles-2 | 67 |
| | Fe(III)-microparticles-2 | 69 |
| | Zr(IV)-microparticles-2 | 65 |
| | Without metal ions | 11 |
| Pureed Banana | Ga(III)-microparticles-2 | 85 |
| | Fe(III)-microparticles-2 | 74 |
| | Zr(IV)-microparticles-2 | 76 |
| | Without metal ions | 42 |

Each value is based upon 2 samples tested, and the standard deviation for all samples was less than 10 percent.

### Example 21

### Extraction and Detection of Bacterial DNA from Spiked Whole Human Blood

A sample preparation method to extract and isolate bacterial DNA from a whole blood matrix may be useful. In this example, a suspension of whole human blood spiked with methicillin-resistant Staphylococcus aureus ATCC #BAA-43 (MRSA) was simultaneously lysed and captured onto Zr(IV)-microparticles-2. After washing and elution, the eluate from the Zr(IV)-microparticles-2 was compared to a control sample via real-time PCR.

Specifically, MRSA was streaked onto non-selective, tryptic soy agar (TSA) media and incubated at 37° C. for 24 hours. Cell suspension was prepared from fresh growth by dilution in TEP buffer (10 mM Tris-HCl, 1 mM EDTA, pH 8.0 and 0.2% PLURONIC L64 (BASF, Mount Olive, N.J.)) using 0.5 McFarland standard corresponding to 1×10⁸ CFU/mL. Serial dilutions were made to obtain different concentrations of bacterial cells.

One hundred (100) μL of appropriate bacterial dilution was added to aliquots of 900 μL of whole human blood to achieve a 1×10² CFU/mL concentration. Two hundred and fifty (250) μL aliquots of spiked whole blood were separated for further processing. Ten (10) μL of Zr(IV)-microparticles-2 (10 mg/mL) and 40 μL of lysostaphin (250 μg/mL, Sigma) were added to each aliquot of spiked whole blood. The bead mixtures were incubated at room temperature for 10 minutes with gentle vortex.

After incubation, the microparticle mixtures were separated with a magnet and 290 μL of each supernatant was removed and discarded (10 μL carryover volume). The microparticles were then washed three times with 90 μL TEP buffer (continuing with 10 μL carryover volume). After the third wash, 10 μL of 20 mg/mL proteinase K (Qiagen, Valencia, Calif.) and 80 μL 20 mM Phosphate, pH 8.5 buffer were added to each sample (100 μL total volume). The mixture was incubated at 65° C. for 10 minutes and then heated at 95° C. for 10 minutes. The heated microparticle mixtures were then separated with a magnet and each supernatant was collected for mecA real-time PCR as described below.

Separately, pure MRSA culture (without whole blood) was extracted and isolated with Zr(IV)-microparticles-2 using a protocol that otherwise followed that above.

Each sample was subjected to real-time PCR amplification for the mecA gene using the following optimized concentrations of primers, probe and enzyme, and thermocycle protocol. The sequence of all primers and probes listed below are given in the 5′→3′ orientation and are known and described in Francois, P., et al., Journal of Clinical Microbiology, 2003, volume 41, 254-260. The forward mecA primer was CATTGATCGCAACGTTCAATTT (SEQ ID NO:1). The mecA reverse primer was TGGTCTTTCTGCATTCCTGGA (SEQ ID NO:2). The mecA probe sequence, TGGAAGTTAGATTGGGATCATAGCGTCAT (SEQ ID NO:3), was dual labeled by 6-carboxyfluorescein (FAM) and IBFQ (IOWA BLACK FQ, Integrated DNA Technologies, Coralville, Iowa) at 5′- and 3′-position, respectively. PCR amplification was performed in a total volume of 10 mL containing 5 mL of sample and 5 mL of the following mixture: two primers (0.5 mL of 10 μM of each), probe (1 mL of 2 μM), MgCl₂ (2 mL of 25 mM) and LightCycler DNA Master Hybridization Probes (1 mL of 10×, Roche, Indianapolis, Ind.). Amplification was performed on the LightCycler 2.0 Real-Time PCR System (Roche) with the following protocol: 95° C. for 30 seconds (denaturation); 45 PCR cycles of 95° C. for 0 seconds (20° C./s slope), 60° C. for 20 seconds (20° C./s slope, single acquisition).

Results were analyzed using the software provided with the Roche LightCycler 2.0 Real Time PCR System. The primers successfully amplified the mecA gene under the conditions presented in this example as shown in Table 4. The results of this experiment indicate that MRSA in whole blood are captured by Zr(IV)-microparticles-2.

TABLE 4
Real-time PCR detection (mecA gene) of MRSA extracted and
isolated from spiked whole blood samples (in duplicate) using
Zr(IV)-microparticles-2 with a microfluidic mimic protocol. Ct
values are reported in duplicate.

| | |
|---|---|
| Sample | Ct |

| | | |
|---|---|---|
| 2.8 × 10² CFU/mL MRSA in | 33.59 | 31.11 |
| whole blood | 32.52 | 31.26 |
| 3.9 × 10² CFU/mL MRSA | 30.13 | 30.76 |
| (pure culture) | | |
| NTC | Negative | Negative |

### Example 22

### Isolation and Detection of Bacterial DNA from Spiked Canine Feces

A sample preparation method to extract and isolate bacterial DNA from a fecal matrix may be useful. In this example, a suspension of canine feces spiked with vancomycin-resistant Enterococcus faecium ATCC #700221 (VRE) was pre-filtered to remove insoluble debris from the sample. VRE in the resulting eluate was then captured onto Zr(IV)-microparticles-2 and lysed on the solid support. After washing and elution, the eluate from the Zr(IV)-microparticles-2 was compared to control samples via real-time PCR.

Specifically, VRE was streaked onto blood agar media and incubated at 37° C. for 20 hours. Cell suspension was prepared from fresh growth by dilution in TEP buffer (10 mM Tris-HCl, 1 mM EDTA, pH 8.0 and 0.2% PLURONIC L64 (BASF, Mount Olive, N.J.)) using 0.5 McFarland standard corresponding to 1×10⁸ CFU/mL.

One-tenth (0.1) g of canine feces was homogenized in 1 mL of 0.1 M 4-morpholineethanesulfonic acid, pH 5.5 (MES) buffer containing 0.1% TRITON X-100 (Sigma-Aldrich, St. Louis, Mo.) by vortex. Ten (10) μL of 1×10⁸ CFU/mL VRE was spiked into the fecal homogenate. The spiked fecal homogenate was briefly vortexed and then filtered through an EMPORE 6065 Filter Plate (3M, St. Paul, Minn.).

Ten (10) μL of 20 mg/mL proteinase K (Qiagen, Valencia, Calif.) and 10 μL of Zr(IV)-microparticles-2 (10 mg/mL) were added to 80 μL of the filtered fecal homogenate. The microparticle mixture was incubated at 37° C. for 10 minutes with 200 rpm shaking and then further incubated at room temperature for 10 minutes with gentle vortex.

After incubation, the sample was separated using a magnet. The supernatant was removed and 100 μL of TEP buffer was added to the sample. The sample was vortexed briefly and reapplied to the magnet. Supernatant was removed and the sample was resuspended in 80 μL of MES buffer.

Ten (10) μL of 12,500 U/mL mutanolysin (Sigma, St. Louis, Mo.) and 10 μL of 25 mg/mL lysozyme (Sigma, St. Louis, Mo.) were added to the sample. The sample was incubated at 37° C. for 10 minutes with 200 rpm shaking and then further incubated at room temperature for 10 minutes with gentle vortex.

After incubation, the microparticle mixture was separated with a magnet and the supernatant was removed and discarded. The microparticles were then washed twice with 100 μL TEP buffer. After the second wash, the microparticles were resuspended in 100 μL of 20 mM Phosphate, pH 8.5 buffer and heated at 95° C. for 10 minutes. The heated microparticle mixture was then separated with a magnet and the supernatant was collected for vanA real-time PCR as described below.

Separately, pure VRE culture (without feces or filtering) was extracted and isolated with Zr(IV)-microparticles-2 using a protocol that otherwise followed that above. Another pure VRE culture (without feces or filtering) was also extracted and isolated with the MagNA Pure LC system using the MagNA Pure LC DNA Isolation Kit III (Bacteria, Fungi) kit (instrument and reagents obtained from Roche, Indianapolis, Ind.) per manufacturer's instructions. The resultant MagNA Pure isolated DNA was then diluted in MES to an equivalent concentration for comparison to the spiked fecal and pure culture samples.

Primers complementary to the vanA gene of vancomycin-resistant Enterococcus faecium are known and described in Volkmann et al., Journal of Microbiological Methods, 2004, volume 56, page 277-286. The forward primer sequence is 5′ CTGTGAGGTCGGTTGTGCG 3′ (SEQ ID NO:7) and the reverse primer sequence is 5′TTTGGTCCACCTCGCCA 3′ (SEQ ID NO:8).

Polymerase chain reaction (PCR) was performed using the LightCycler FastStart DNA Master SYBR Green I kit (Roche, Indianapolis, Ind.). Fourteen microliters (14 μL) of enzyme was added to one tube of reaction buffer. The enzyme/reaction buffer mixture was vortexed and PCR reactions were created in LightCycler capillaries using the following recipe per reaction: 9 μL PCR-grade H₂O, 1 μL of 10 μM forward primer, 1 μL of 10 μM reverse primer, 4 μL enzyme/reaction buffer mix, and 5 μL sample DNA.

Reactions were placed into the Roche LightCycler 2.0 Real-Time PCR System and the following thermocycle profile was applied to the samples: 95° C. for 10 minutes followed by 45 cycles of the following three steps in order, 95° C. for 10 seconds (20° C./s slope), 50° C. for 10 seconds (20° C./s slope) and 72° C. (20° C./s slope, acquisition) for 30 seconds.

Results were analyzed using the software provided with the Roche LightCycler 2.0 Real Time PCR System. The primers successfully amplified the vanA gene under the conditions presented in this example as shown in Table 5. The results of this experiment indicate that VRE in feces are captured by Zr(IV)-microparticles-2 after a pre-filtration step.

TABLE 5
Real-time PCR detection (vanA gene) of VRE extracted and isolated
from spiked canine fecal samples (in quadruplicate) using filtration
and Zr(IV)-microparticles-2. Ct values are reported in duplicate.

| | |
|---|---|
| Sample | Ct |

| | | |
|---|---|---|
| 10⁵ CFU/mL VRE in feces, filtered | 28.77 | 29.47 |
| | 30.30 | 28.60 |
| | 27.60 | 27.81 |
| | 27.44 | 26.89 |
| 10⁵ CFU/mL VRE (pure culture) | 22.14 | 23.74 |
| MagNA Pure VRE DNA | 20.62 | 20.99 |
| (gc/mL equivalent to 10⁵ CFU/mL) | | |

All references and publications or portions thereof cited herein are expressly incorporated herein by reference in their entirety into this disclosure. Exemplary embodiments of this invention are discussed and reference has been made to some possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the embs provided below and equivalents thereof.

1. A composition comprising:
an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and
at least one double stranded polynucleotide bound to at least one of the metal ions, M^{y+};
wherein M is selected from the group consisting of zirconium, gallium, iron, aluminum, scandium, titanium, vanadium, yttrium, and a lanthanide; y is an integer from 3 to 6; and x is 1 or 2.

2. A composition comprising:
an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and
at least one polynucleotide bound to at least one of the metal ions, M^{y+};
wherein M is selected from the group consisting of zirconium, gallium, iron, aluminum, scandium, titanium, vanadium, yttrium, and a lanthanide; y is an integer from 3 to 6; and x is 1 or 2; and
wherein the composition has a pH of 4.5 to 6.5.

3. The composition of claim 1, wherein M^{y+} is Zr⁴⁺ or Ga³⁺.

4. A method of separating and optionally assaying at least one double stranded polynucleotide from a sample material comprising:
providing an immobilized-metal support material comprising a substrate having a plurality of —C(O)O⁻ or —P(O)(—OH)₂₋ₓ(—O⁻)ₓ groups bound to the substrate and a plurality of metal 1 ions, M^{y+}, bound to the —C(O)O⁻ or —P(O)(—OH)₂₋ₓ(—O⁻)ₓ groups; and
contacting the sample material with the plurality of metal ions, M^{y+}, bound to the —C(O)O⁻ or —P(O)(—OH)₂₋ₓ(—O⁻)ₓ groups to provide a composition comprising a) the at least one double stranded polynucleotide bound to the immobilized-metal support material and b) a supernate comprising the sample material having a reduced amount of the at least one double stranded polynucleotide; and
separating a) the at least one double stranded polynucleotide bound to the immobilized-metal support material from b) the supernate comprising the sample material having a reduced amount of the at least one double stranded polynucleotide;
wherein M is selected from the group consisting of zirconium, gallium, iron, aluminum, scandium, titanium, vanadium, yttrium, and a lanthanide; y is an integer from 3 to 6; and x is 1 or 2.

5. A method of separating and optionally assaying at least one polynucleotide from a sample material comprising:
providing an immobilized-metal support material comprising a substrate having a plurality of —C(O)O⁻ or —P(O)(—OH)₂₋ₓ(—O⁻)ₓ groups bound to the substrate and a plurality of metal 1 ions, M^{y+}, bound to the —C(O)O⁻ or —P(O)(—OH)₂₋ₓ(—O⁻)ₓ groups; and
contacting the sample material with the plurality of metal ions, M^{y+}, bound to the —C(O)O⁻ or —P(O)(—OH)₂₋ₓ(—O⁻)ₓ groups, at a pH of 4.5 to 6.5, to provide a composition comprising a) the at least one polynucleotide bound to the immobilized-metal support material and b) a supernate comprising the sample material having a reduced amount of the at least one polynucleotide; and
separating a) the at least one polynucleotide bound to the immobilized-metal support material from b) the supernate comprising the sample material having a reduced amount of the at least one polynucleotide;
wherein M is selected from the group consisting of zirconium, gallium, iron, aluminum, scandium, titanium, vanadium, yttrium, and a lanthanide; y is an integer from 3 to 6; and x is 1 or 2; and
wherein the composition has a pH of 4.5 to 6.5.

6. The method of claim 4, wherein the sample material includes a plurality of cells, viruses, or a combination thereof; wherein the sample material is contacted with a lysis reagent when contacting the sample material with the plurality of metal ions, MY bound to the —C(O)O⁻ or —P(O)(—OH)₂₋ₓ(—O⁻)ₓ groups; and further comprising lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide.

7. The method of claim 5, wherein the sample material includes a plurality of cells, viruses, or a combination thereof; wherein the sample material is contacted with a lysis reagent when contacting the sample material with the plurality of metal ions, M^{y+}, bound to the —C(O)O⁻ or —P(O)(—OH)₂₋ₓ(—O⁻)ₓ groups; and further comprising lysing the cells, viruses, or a combination thereof to provide the composition comprising a) the at least one polynucleotide bound to the immobilized-metal support material and b) the supernate comprising the sample material having a reduced amount of the at least one polynucleotide.

8. The method of claim 4, wherein the sample material includes a plurality of cells, viruses, or a combination thereof; wherein contacting the sample material with the plurality of metal ions, M^{y+}, bound to the —C(O)O⁻ or —P(O)(—OH)₂₋ₓ(—O⁻)ₓ groups provides a) at least a portion of the plurality of cells, viruses, or a combination thereof bound to the immobilized-metal support material and b) a supernate comprising the sample material having a reduced number of cells, viruses, or a combination thereof; and further comprising separating the supernate comprising the sample material having a reduced number of cells, viruses, or a combination thereof from the at least a portion of the plurality of cells, viruses, or a combination thereof bound to the immobilized-metal support material.

9. The method of claim 5, wherein the sample material includes a plurality of cells, viruses, or a combination thereof; wherein contacting the sample material with the plurality of metal ions, M^{y+}, bound to the —C(O)O⁻ or —P(O)(—OH)₂₋ₓ(—O⁻)ₓ groups provides a) at least a portion of the plurality of cells, viruses, or a combination thereof bound to the immobilized-metal support material and b) a supernate comprising the sample material having a reduced number of cells, viruses, or a combination thereof; and further comprising separating the supernate comprising the sample material having a reduced number of cells, viruses, or a combination thereof from the at least a portion of the plurality of cells, viruses, or a combination thereof bound to the immobilized-metal support material.

10. The method of claim 9, further comprising assaying the cells, viruses, or a combination thereof bound to the immobilized-metal support material.

11. The method of claim 8, further comprising adding a lysis reagent to the at least a portion of the plurality of cells, viruses, or a combination thereof bound to the immobilized-metal support material and lysing the cells, viruses, or a combination thereof to provide the composition comprising a) the at least one double stranded polynucleotide bound to the immobilized-metal support material and b) the supernate comprising the sample material having a reduced amount of the at least one double stranded polynucleotide.

12. (canceled)

13. The method of claim 9, further comprising adding a lysis reagent to the at least a portion of the plurality of cells, viruses, or a combination thereof bound to the immobilized-metal support material and lysing the cells, viruses, or a combination thereof to provide the composition comprising a) the at least one polynucleotide bound to the immobilized-metal support material and b) the supernate comprising the sample material having a reduced amount of the at least one polynucleotide.

14-15. (canceled)

16. The method of claim 4, further comprising amplifying the at least one double stranded polynucleotide bound to the immobilized-metal support material to provide a plurality of amplicons.

17. The method of claim 5, further comprising amplifying the at least one polynucleotide bound to the immobilized-metal support material to provide a plurality of amplicons.

18. A device for processing sample material, the device having:
at least one first chamber capable of containing or channeling a fluid, wherein the at least one first chamber contains a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and
at least one second chamber separate from the first chamber and capable of receiving and containing the fluid, the immobilized-metal support material, or both from the at least one first chamber;
wherein M is selected from the group consisting of zirconium, gallium, iron, aluminum, scandium, titanium, vanadium, yttrium, and the lanthanides; y is an integer from 3 to 6; and x is 1 or 2.

19. A kit for separating at least one polynucleotide from a sample material, the kit comprising:
a device having at least one chamber capable of containing or channeling a fluid;
an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; wherein M is selected from the group consisting of zirconium, gallium, iron, aluminum, scandium, titanium, vanadium, yttrium, and the lanthanides; y is an integer from 3 to 6; and x is 1 or 2; and
at least one reagent selected from the group consisting of a lysis reagent, a lysis buffer, a binding buffer, a wash buffer, and an elution buffer.

20. A composition comprising:
an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups; and
a plurality of microorganisms, selected from the group consisting of bacterial cells, yeast cells, mold cells, viruses, and a combination thereof, non-specifically bound to the immobilized-metal support material;
wherein M is selected from the group consisting of zirconium, gallium, iron, aluminum, scandium, titanium, vanadium, yttrium, and a lanthanide; y is an integer from 3 to 6; and x is 1 or 2.

21. A method of isolating microorganisms comprising:
providing a composition comprising an immobilized-metal support material comprising a substrate having a plurality of —C(O)O⁻ or —P(O)(—OH)₂₋ₓ(—O⁻)ₓ groups bound to the substrate and a plurality of metal ions, M^{y+}, bound to the —C(O)O⁻ or —P(O)(—OH)₂₋ₓ(—O⁻)ₓ groups;
providing a sample suspected of having a plurality of microorganisms selected from the group consisting of bacterial cells, yeast cells, mold cells, viruses, and a combination thereof; and
contacting the composition with the sample; wherein at least a portion of the plurality of microorganisms from the sample become non-specifically bound to the immobilized-metal support material;
separating the immobilized-metal support material from the remainder of the sample after the at least a portion of the plurality of microorganism from the sample become non-specifically bound to the immobilized-metal support material
wherein M is selected from the group consisting of zirconium, gallium, iron, aluminum, scandium, titanium, vanadium, yttrium, and a lanthanide; y is an integer from 3 to 6; and x is 1 or 2.

22. (canceled)

23. The method of claim 21, further comprising detecting the at least a portion of the plurality of microorganisms.

24-25. (canceled)

26. The method of claim 21, wherein the sample is selected from the group consisting of a clinical sample, a food sample, and an environmental sample.

27. The composition of claim 2, wherein M^{y+} is Zr⁴⁺ or Ga³⁺.

28. The method of claim 8, further comprising assaying the cells, viruses, or a combination thereof bound to the immobilized-metal support material.
